(19) United States
(12) Patent Application Publication
(10) Pub. No.: US 2011/0052660 A1
(43) Pub. Date: Mar. 3, 2011

(54) CERAMIC SCAFFOLDS FOR BONE REPAIR

(75) Inventors: Yunzhi YANG, Houston, TX (US); Yongxing LIU, Mishawaka, IN (US)

(73) Assignee: BOARD OF REGENTS OF THE UNIVERSITY OF TEXAS SYSTEM, Austin, TX (US)

(21) Appl. No.: 12/902,886

(22) Filed: Oct. 12, 2010

Related U.S. Application Data
(63) Continuation-in-part of application No. 12/074,434, filed on Mar. 4, 2008.
Application No. 12/074,434 is a continuation-in-part of application No. PCT/US09/03501, filed on Jun. 10, 2009.
(60) Provisional application No. 61/250,151, filed on Oct. 9, 2009.
(60) Provisional application No. 60/918,434, filed on Mar. 16, 2007.
(60) Provisional application No. 61/131,810, filed on Jun. 12, 2008.

(51) Int. Cl. A61K 9/00; A61P 19/00; B29C 44/04

(52) U.S. Cl. 424/426; 264/45.1

(57) ABSTRACT

Ceramic articles as functional biodegradable scaffolds with graded porosity are made by a process that includes a hardening step in which the liquid of a liquid-containing ceramic composition is extracted from the ceramic composition by exposing the ceramic composition to a solvent in which the liquid in the composition is soluble before the ceramic composition is solidified into the final ceramic article. An exemplary calcium phosphate porous ceramic article constructed in accordance with the process is useful as an implant to repair a bone defect.

### CROSS-REFERENCE TO RELATED APPLICATIONS

This application is a continuation-in-part of PCT/US2009/003501 filed Jun. 10, 2009, from which priority under 35 U.S.C. §120 is claimed. PCT/US2009/003501 claims priority of U.S. Provisional Patent Application No. 61/131,810 filed Jun. 12, 2008. This application also claims the benefit under 35 U.S.C. §119(e) of U.S. Provisional Patent Application No. 61/250,151 filed Oct. 9, 2009. This application is also a continuation-in-part of co-pending U.S. patent application Ser. No. 12/074,434 filed Mar. 4, 2008, which claims the benefit under 35 U.S.C. §119(e) of U.S. Provisional Patent Application No. 60/918,434 filed Mar. 16, 2007. The disclosures of those applications are hereby incorporated herein by reference.

### TECHNICAL FIELD

The present disclosure pertains to the field of fabricating ceramic articles and particularly to the field of fabricating porous ceramic articles which may be used for various purposes, such as a scaffolding for many different applications, such as for tissue engineering and bone replacement and repair. In one particular embodiment, the invention pertains to the field of biodegradable ceramic scaffolds, such as calcium phosphate based scaffolds, that are useful in the treatment of skeletal defects.

### BACKGROUND

Ceramics are used extensively in a large number of industrial applications. They are used as building materials, as cements and mortars, as abrasives, and in recent years ceramics have been developed for specialized uses in such fields as electronics, communications, and medicine.

In medicine, biodegradable macroporous ceramic scaffolds have been used as engineered grafts for tissue engineering, particularly bone tissue engineering. Such scaffolds typically are made with hydroxyapatite (HA) or tricalcium phosphate (TCP), or a combination of HA and TCP, with additives such as silica, magnesium, sodium, potassium, and zinc. The porous nature of these scaffolds permits the ingrowth of vascular and structural tissues and, because the scaffolds are biodegradable, can be used safely and without the need to remove the implant from the body.

For bone repair, particularly for defects in the spine and long bones, such as the bones of the legs, it is critically important that a ceramic scaffold implant have a high compressive strength and that this strength is maintained as the implant is biodegraded before the bone itself has healed and has sufficient strength. However, there is an inverse relationship between porosity and mechanical strength of the implants as the mechanical strength decreases as the porosity and pore size increases. In addition, biodegradable synthetic bone implants decrease in strength as the implant is degraded by contact with body fluids. Loss of strength of an implant at a time before the healed bone is able to support weight or support itself can lead to failure of the implant and of the repair process.

Ma, U.S. Pat. No. 6,673,285 discloses a method for fabrication of porous articles, such as polymer scaffolds. Ma discloses that the scaffolds may be made by casting a composition onto a negative replica of a desired macroporous architecture of the porous article to form a body, and that the negative replica, referred to as a porogen, is removed, thereby forming the porous article. Ma discloses that this method may be utilized to form a porous article from various materials, including polymers, ceramics, glass, and inorganic compounds

Various scientific articles describe methods of manufacture of macroporous ceramic (CaP) scaffolds of various porosity and report on the compressive strength of these scaffolds. See, Hing, J. Mater. Sci. Mater. Med., 10(3):135-145 (1999); Liu, Ceramics International, 23:135-139 (1997); Seplveda, J. Biomed. Mater. Res., 50:27-34 (2000); Ramay, Biomaterials, 24:3293-3302 (2003); Almirall, Biomaterials, 25:3671-3680 (2004); Cyster, Biomaterials, 26:697-702 (2005); Silva, Biomaterials, 27:5909-5917 (2006); Uemura, Biomaterials, 24:2277-2286 (2003); Sous, Biomaterials, 19:2147-2153 (1998); Guo, Tissue Engineering, 10:1830-1840 (2004); Kwon, J. Am. Ceramic Soc., 85:3129-3131 (2002); and Milosevski, Ceramics International, 25:693-696 (1999). These reports show that the strength of porous CaP scaffolds tends to decrease with increasing porosity and that most of the scaffolds produced by the prior art methods have a compressive strength of only about 0.8 to 8 MPa (megapascals) with one report of a scaffold having 70% porosity, pores not completely interconnected, and a compressive strength of about 11 MPa.

Large bone defects that result from disease or damage can be replaced or reconstructed by a structural graft or prosthesis. Use of a patient's own bone as the source of a graft, referred to as an autograft, remains the “gold standard” of graft choice due to its excellent osteogenicity, osteoinductivity, and osteoconductivity. However, the use of autografts is limited in clinical situations by the lack of available bone for harvest, particularly in the case of children and large-scale defects, significant postoperative morbidity at donor sites, increased operative time and blood loss, and additional cost. An alternative to autografts is the use of bone from another individual, referred to as an allograft. However, the preparation of an allograft requires donor screening, sterile harvesting, and processing, and presents an increased risk of infections and disease transmission, as well as inconsistency in quality. As a result of these problems, biomimetic synthetic bone grafts are desirable.

Calcium phosphate (CaP) ceramics are attractive alternatives for artificial bone scaffold construction. CaP is the main inorganic component of vertebrate calcified hard tissues. The CaP materials used most frequently in clinical settings are beta-tricalcium phosphate (TCP), hydroxyapatite (HA) and their composites. The degradation of CaP by dissolution does not produce any known harmful effects. Sterilization and shelf storage of the materials do not present difficulties and there is no risk of disease transmission or of an immunogenic response. Additionally, CaP scaffolds can be used to deliver living cells and growth factors to the implantation site.

It is of critical importance that the CaP scaffold has a macroporous structure to permit bone growth into and onto the scaffold. Conventional techniques for fabricating 3-dimensional CaP scaffolds include foaming, sacrificial templates, replication of polymer foams by infiltration with CaP slurries, hydrothermal conversion of either coral or bone, and replamineform. However, the resulting porous structures are typically rather random in architectures with regards to pore sizes, shapes, alignment, and interconnectivity. Robocasting, a solid freeform fabrication technique, has been developed to fabricate HA scaffolds and show potential for better controlling pore size, shape and a customized fabrication. However, this method requires expensive 3D freeform manufacturing systems and special CaP ceramic slurries for the machine. Consequently, this method has not been widely adopted.

A significant need remains for a method for producing a CaP scaffold for bone repair applications that provides control over the architecture and composition of the scaffold and that can be used to provide a scaffold that mimics the physical and chemical properties of bone.

### SUMMARY

In accordance with certain embodiments, a method of making a ceramic article is provided. The method generally comprises a) forming at least one ceramic composition containing a ceramic material (e.g., calcium phosphate) and a liquid (e.g., water) into a defined shape comprising at least two zones with different porosity or pore size, wherein a second zone surrounds a first zone in at least two dimensions (e.g., along the x and y axis of a three-dimensional article). The method also includes exposing the shaped ceramic composition(s) to a solvent (e.g., an alcohol) in which the liquid is soluble or miscible, thereby removing the liquid from and hardening the shaped ceramic composition. The method further includes solidifying the hardened ceramic composition(s), to provide the ceramic article. In some embodiments, in a) said forming comprises casting at least one said composition onto a template or replica that is insoluble in the solvent. For example, in some applications the replica is a negative replica comprising a sacrificial porogen comprising a multiplicity of discrete elements (e.g., small wax beads). The elements of the sacrificial porogen are organized into at least two said zones that differ based on porogen size, in some embodiments.

In some embodiments, before casting a ceramic composition onto a replica, the multiplicity of discrete elements are caused to coalesce to a degree that corresponds to interconnectivity of pores of at least 70% in the ceramic article. For example, a multiplicity of small wax beads are slightly melted to form the interconnections between adjacent beads that will be converted to interconnected pores in the final ceramic articles.

In some embodiments, a disclosed method includes forming a first composition into a first zone; and forming a second composition into a second zone that surrounds the first zone in at least two dimensions. The first and second zones comprise respective first and second multiplicities of discrete elements wherein the discrete elements of the first zone differ in size from the discrete elements of the second zone, to provide the ceramic article with graded porosity. In some embodiments of the above-described methods, each said zone has a defined shape and the resulting ceramic article has a stepwise graded porosity from one zone to another. In some embodiments two or more zones together form a substantially continuous gradation of porosity. In some embodiments, the porosity of the ceramic article is graded laterally or radially, and in some embodiments the porosity is graded vertically.

In some embodiments, a disclosed method includes at least partially removing a sacrificial porogen from the hardened ceramic composition before solidifying the hardened ceramic composition. In some embodiments, a disclosed method includes exposing a shaped ceramic composition to stepwise increases in solvent concentration to harden said composition.

In some embodiments, a disclosed method also includes associating a polymer, or a growth factor, or both, with the solidified ceramic article. In some embodiments, an above-described method includes forming a first ceramic composition into a first defined zone; forming a second ceramic composition into a second defined zone that surrounds the first defined zone in at least two dimensions; and forming a third ceramic composition into a third defined zone that surrounds the second defined zone in at least two dimensions. The first, second and third zones are concentric in some embodiments. In some embodiments, the ceramic compositions used to form first, second and third compositions, for forming respective first, second and third zones, differ from each other. In some embodiments, at least two of the ceramic compositions are the same.

Also provided in accordance with certain embodiments is a ceramic article comprising at least two zones comprising at least one ceramic material, wherein a second zone surrounds a first zone in at least two dimensions. At least two of the zones have different porosity or pore size, and have solid struts between pores and at least 70% pore interconnectivity. In some embodiments, the ceramic article has compressive strength equal to or exceeding that of cortical bone. In some embodiments, the ceramic material comprises calcium phosphate (e.g., hydroxyapatite, tricalcium phosphate, or a mixture of hydroxyapatite or tricalcium phosphate, or any other suitable form of calcium phosphate). In some embodiments, a ceramic article also contains a polymer or a bone growth factor, or both.

In some embodiments, a ceramic article is made by an above-described process. In some embodiments, a disclosed ceramic article comprises a first or innermost zone having a porosity in the range of about 70% to about 100% and mean pore diameter in the range of about 1 μm to about 1 cm; a third or outermost zone having a porosity in the range of about 70% to about 90% and mean pore diameter in the range of about 1 μm to about 1 cm; and a second or middle zone disposed between and in contact with said innermost and outermost zones and having a greater density than that of at least one of the innermost and outermost zones. In some embodiments, the innermost zone has a porosity in the range of about 70% to about 90% and a mean pore diameter in the range of about 300-500 μm, the middle zone has a porosity of about 20%, and the outermost zone has a porosity in the range of about 70% to about 90% and a mean pore diameter in the range of about 1 μm to about 2 cm.

Also provided in accordance with certain embodiments is a method of repairing a bone defect in an individual, comprising implanting into a defect of a bone within the individual an above-described ceramic article configured as a scaffold that comprises at least 70% porosity, and allowing bony tissue to grow in the implanted scaffold while the ceramic article gradually biodegrades. The bone defect may be the result of an injury or caused by a disease, for example.

These and other embodiments, features and advantages will be apparent with reference to the following description and drawing.

### BRIEF DESCRIPTION OF THE DRAWING

FIG. 1 is a photograph showing, on the left side, a ceramic composition slurry in a plastic tube container prior to drying, in the middle, a green body dried by the solvent extraction step in accordance with an embodiment of the present invention, and on the right side, a green body dried by exposure to air at room temperature without the solvent extraction step.

FIG. 2 is a graph comparing the compressive strength in MPa of a porous ceramic article made by a method according to an embodiment of the invention, with porous ceramic articles made by other methods. The arrows point to data points for the porous ceramic articles made by a method of the embodiment.

FIG. 3A is a 3-dimensional computer-reconstructed Micro CT (computed tomography) image of a dense scaffold showing the lack of pores made to mimic the structure of cortical bone. FIG. 3B is a top view 2-dimensional Micro CT image of the dense scaffold. FIG. 3C is a side view 2-dimensional Micro CT image of the dense scaffold. FIG. 3D is a 3-dimensional computer-reconstructed Micro CT image of a two-zone graded ceramic scaffold having pores in the inner zone and lacking pores in the outer zone made to mimic the structure of bone. FIG. 3E is a top view 2-dimensional Micro CT image of the graded scaffold. FIG. 3F is a side view 2-dimensional Micro CT image of the graded scaffold.

FIG. 4A is a 3-dimensional computer-reconstructed Micro CT image of a porous scaffold with pores of 600 μm to 800 μm. FIG. 4B is a top view 2-dimensional Micro CT image of the porous scaffold. FIG. 4C is a side view 2-dimensional Micro CT image of the porous scaffold. FIG. 4D is a 3-dimensional computer-reconstructed Micro CT image of a porous scaffold with pores of 350 μm to 500 μm. FIG. 4E is a top view 2-dimensional Micro CT image of the porous scaffold. FIG. 4F is a side view 2-dimensional Micro CT image of the porous scaffold.

FIG. 5 is a scanning electron microscopy photograph showing the solid struts and interconnectivity between pores of a scaffold made by a negative replica method. The black arrows indicate the solid struts. Pores are indicated by dashed white arrows, and interconnecting pores are indicated by solid white arrows.

FIG. 6A is a 3-dimensional computer-reconstructed Micro CT image of a radially graded porous ceramic article in which an inner zone of the article contains pores between 350 μm to 500 μm in diameter and an outer zone contains pores between 600 μm and 800 μm. FIG. 6B is a top view 2-dimensional Micro CT image of this radially graded porous ceramic article. FIG. 6C is a corresponding Micro CT side image.

FIG. 7A is a 3-dimensional computer-reconstructed Micro CT image of a radially graded porous ceramic article in which an inner zone of the article contains pores between 600 μm and 800 lam in diameter and an outer zone contains pores between 350 μm to 500 μm in diameter. FIG. 7B is a 2-dimensional Micro CT top to bottom image of this radially graded porous ceramic article. FIG. 7C is a corresponding 2-dimensional Micro CT side image.

FIG. 8A is a 2-dimensional Micro CT side image of a vertically graded macroporous ceramic article in which the top portion has smaller pores of 300 μm to 400 μm and the bottom portion has larger pores of 600 μm to 700 μm. FIG. 8B is a top view 3-dimensional computer-reconstructed Micro CT image of the vertically graded macroporous article showing the smaller pores at the top surface. FIG. 8C is a bottom view 3-dimensional computer-reconstructed Micro CT image of the article showing the larger pores at the bottom surface.

FIG. 9A is a scanning electron microscopy photograph of a compositionally graded porous ceramic article. FIG. 9B is a graph that indicates the varying composition of the article at various numbered locations as shown in FIG. 9A.

FIG. 10 is a graph showing the dissolution behaviors of porous TCP scaffolds following immersion in Tris buffer for 4 weeks. A shows the dissolution behavior of the scaffolds with uniform 600-800 μm pores. B shows the dissolution behavior of the scaffolds with uniform 350-500 μm pores. C shows the dissolution behavior of the graded scaffolds with central 350-500 μm pores and peripheral 600-800 μm pores. D shows the dissolution behavior of the graded scaffolds with central 600-800 μm pores and peripheral 350-500 μm pores.

FIG. 11 is a series of photographs showing the morphological changes of graded CaP scaffolds that occurred in vitro. C1 is a graded scaffold with central 350-500 μm pores and peripheral 600-800 μm pores. C2 is the scaffold of C1 following immersion in acidic buffer medium at pH3. D1 is a graded scaffold with central 600-800 μm pores and peripheral 350-500 μm pores. D2 is the scaffold of D1 following immersion the acidic buffer medium.

FIG. 12 is a non-decalcified histological examination of CaP scaffolds showing morphology changes that occur following subcutaneous implantation of the scaffolds for a period of one month. A shows results observed for the scaffold with uniform large pores of 600-800 μm. B shows results observed for the scaffold with uniform small pores of 350-500 μm. C shows results observed for the scaffold with graded pores having central small pores of 350-500 μm and peripheral large pores of 600-800 μm. D shows results observed for the scaffold with graded pores having central larges pores of 600-800 μm and peripheral small pores of 350-500 μm.

FIG. 13 is a graph showing the initial loading of BMP-2 onto scaffolds of different pore sizes. * indicates significant differences (P<0.05).

FIG. 14 is a graph showing the cumulative elution of BMP-2 from scaffolds of different pore sizes.

FIG. 15 shows BMP-2 induced ectopic bone formation in non-decalcified porous CaP scaffolds at one month after implantation. A1, B1, C1 and D1 are micro CT images; A2, B2, C2 and D2 are histology pictures obtained with Anderson's rapid bone stain counterstained with acid fuchsin. A1 and A2 are of a scaffold with uniform 600-800 μm large pores. B1 and B2 are of a scaffold with uniform 350-500 μm pores. C1 and C2 are of a graded scaffold with central 350-500 μm pores and peripheral 600-800 μm pores. D1 and D2 are of a graded scaffold with central 600-800 μm pores and peripheral 350-500 μm pores.

FIG. 16 shows radiographs taken at 2 weeks (A) and 4 weeks (B) after implantation of a CaP scaffold constructed by a method of the invention into a defect in the radius. Healing of the radial defect is apparent after two weeks and after four weeks.

FIG. 17 shows micro CT images of healing of a defect of the radius following implantation with a CaP scaffold constructed by a method according to an embodiment of the invention. A represents a cross-sectional view. B represents a longitudinal view.

FIG. 18 shows micro CT images of a scaffold comprising a functional gradient having a central porous zone (C), a middle dense zone (M), and a peripheral porous zone (P). (A) Coronal view of 2D image. (B) Sagital view of 2D image. The central porous zone is with 80% porosity and has macropores of 300-500 μm. The middle dense zone is with 20% porosity. The peripheral porous zone is with 80% porosity and has macropores of 600-800 μm.

### DETAILED DESCRIPTION

It is to be understood that both the general and detailed descriptions are exemplary and explanatory only, and are not restrictive of the invention, as claimed. In this application, the use of the singular includes the plural, the word “a” or “an” means “at least one,” and the use of “or” means “and/or,” unless specifically stated otherwise. Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

Temperatures, ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range, as if each numerical value and sub-range is explicitly recited. For example, a concentration range of 70 vol. % to 95 vol. % should be interpreted to include not only the express limits of 70 vol. % and 95 vol. %, but also to include every intervening value such as 75, 82 and 90 vol % and all sub-ranges such as 80-90 vol. %, and so forth.

The term “about” when referring to a numerical value or range is intended to include larger or smaller values resulting from experimental error that can occur when taking measurements. Such measurement deviations are usually within plus or minus 10 percent of the stated numerical value. Any use of the term “optionally” with respect to any element of a claim is intended to mean that the subject element is required, or alternatively, is not required. Both alternatives are intended to be within the scope of the claim. Other terms that are used in this disclosure and in the claims are defined elsewhere herein.

It was discovered that removing a portion or all of the liquid present in a fluid ceramic composition by extraction with a solvent having a lower surface tension than the liquid, thereby obtaining a hardened ceramic composition, followed by solidifying the resultant hardened ceramic composition, such as by the application of heat, results in a ceramic article possessing unexpectedly higher strength than that possessed by similar ceramic articles that are made without the solvent-based liquid removal step. Related ceramic articles and their methods of making are described in International Patent PCT/US2009/03501, the disclosure of which is hereby incorporated herein by reference.

Some embodiments of the presently disclosed methods apply and extend the aforesaid discovery, provide for the manufacture of strong ceramic articles suitable for use in various industries, such as for medical devices, building construction, electronics, telecommunications, and in the manufacture of housewares. Some embodiments of the methods described herein are especially useful in the manufacture of ceramic articles for implantation into the body of a human or another mammal. In some embodiments, the articles contain biocompatible and/or biodegradable materials. For example, some embodiments of the ceramic articles are used as porous implants such as those used for bone reconstruction and regeneration techniques.

An exemplary method of making a ceramic article generally includes forming a fluid ceramic composition containing a liquid into a desired shape, exposing the resulting intermediate structure to a solvent in which the liquid of the ceramic composition is soluble at a concentration and for a time sufficient to extract at least a portion of the liquid from the composition. In some cases, most or all of the liquid is removed in this manner. Typically, but not necessarily, the liquid from the composition is replaced by an equal volume of the solvent. Following the extraction, the resultant “dried” composition, is caused or permitted to solidify to form a ceramic article with improved structural properties compared to similar ceramic articles not made by this method.

The resulting ceramic articles are sometimes referred to herein as “solvent-hardened,” which indicates that, prior to solidifying to form the ceramic article, the fluid ceramic composition that was used to make the article was exposed to a solvent in which liquid in the composition was soluble at a concentration and for a time sufficient to extract the liquid from the composition and, following this extraction, the composition was caused or permitted to solidify to form the solvent-hardened ceramic article.

In some embodiments, a method is provided for making a macroporous CaP scaffold having high interconnectivity and mechanical strength, compared to CaP scaffolds made by other methods. In some applications, a CaP scaffold is made by a negative replica method, using a negative replica that is defined by a template comprising a multiplicity of discrete porogen particles. In some instances, it is preferred that a hardening step utilizing an extraction solvent is performed prior to the final curing of the scaffold, typically prior to removal of the negative template from the ceramic composition. An illustrative preferred method for making this type of macroporous CaP scaffold is described in Example 1B below.

In another embodiment, a method of making a CaP scaffold uses a negative replica method which includes a hardening step utilizing an extraction solvent that is performed prior to the final curing of the scaffold.

In accordance with another embodiment, a method of treating a skeletal defect in a human or other mammal is provided, in which an above-described macroporous CaP scaffold is implanted into or onto a bone within the body of the individual in need thereof. The implanted scaffold is permitted to remain in place in or on the bone for a time sufficient for new bone to develop on the scaffold.

As used herein, the term “removing” when referring to a liquid of a fluid ceramic composition refers to reducing the concentration of the liquid in the ceramic composition. The removing may be accompanied by replacement of the volume of liquid removed with a smaller, equivalent, or higher volume of another liquid.

As used herein, the term “extract” when referring to the liquid of a fluid ceramic composition, means to reduce the concentration of the liquid in the ceramic composition by exposing the ceramic composition containing the liquid to a solvent in which the liquid of the ceramic composition is soluble. Such extraction is preferably performed by immersing a container containing the ceramic composition into a larger container containing the solvent. This method of extraction typically, but not necessarily, results in a dilution of the concentration of the liquid in the ceramic composition by providing a larger volume into which the liquid will dissolve. Generally, but not necessarily, the volume of the liquid that is removed from the ceramic composition will be replaced by the solvent, which is more easily removed (e.g., volatile). The extraction may also be performed by any other method by which a liquid may be extracted by use of a solvent in which the liquid is soluble. Examples include pouring the solvent into the container containing the ceramic composition, or by spraying.

As used herein, the term “ceramic material” refers to an inorganic non-metallic crystalline or partly crystalline, or glass, material that either solidifies upon cooling from a molten mass or that forms a solid structure due to the action of heat. Any suitable ceramic material may be used in the disclosed methods and articles. Some non-limiting examples are aluminum silicates, zirconium oxides such as zirconium dioxide, aluminum oxides, titanium oxides, tantalum oxides, carbides, borides, nitrites, and silicides, calcium ceramics such as calcium nitrite, calcium sulfate, calcium hydrogen sulfate, calcium hydroxide, calcium carbonates, calcium hydrogen carbonate, and calcium phosphates, alkali metal hydroxides, alkaline earth hydroxides, disodium hydrogen phosphate, disodium hydrogen phosphate dodecahydrate, disodium hydrogen phosphate heptahydrate, sodium phosphate dodecahydrate, dipotassium hydrogen phosphate, potassium phosphate tribasic, diammonium hydrogen phosphate, ammonium phosphate trihydrate, sodium bicarbonate, barium titanate, bismuth strontium calcium copper oxide, boron carbide, boron nitride, ferrite, lead zirconate titanate, magnesium diboride, silicon carbide, silicon nitride, steatite, uranium oxide, yttrium barium copper oxide, and zinc oxide.

As used herein, the term “ceramic article” refers to an article of manufacture that is made from a ceramic material. A ceramic article has a glazed or unglazed body of crystalline or partly crystalline structure, or of glass, which body is produced from essentially inorganic non-metallic substances and is either formed from a molten mass that solidifies upon cooling or is formed and simultaneously or subsequently matured by the action of heat.

As used herein the term “ceramic composition” refers to a composition comprising a ceramic material that flows sufficiently for casting purposes. The ceramic composition may be a solution or a non-solution and may be, for example, in the form of a melt, a slurry, or a flowable paste, which may be made by wetting a powder of a ceramic material with a liquid. The ceramic composition may contain additional components, such as binders, plasticizers, anti-flocculants, and lubricants.

The liquid of the fluid ceramic composition may be any liquid or combination of liquids into which a ceramic material may be dispersed, with or without the use of additional materials such as a binder, plasticizer, anti-flocculant, or lubricant. In some embodiments, the ceramic composition preferably includes a binder, which is typically a polymer, which may be water miscible or immiscible, and which may be hydrophilic, hydrophobic, or amphiphilic. Non-limiting examples of water soluble binders include polyvinylpyrrolidones (PVP), polyvinylpyrrolidone/vinyl acetate copolymers, polyvinyl alcohols (PVA), carboxymethyl celluloses, hydroxypropyl cellulose starches, polyethylene oxides (PEO), polyacrylamides, polyacrylic acids, cellulose ether polymers, polyethyl oxazolines, esters of polyethylene oxide, esters of polyethylene oxide and polypropylene oxide copolymers, urethanes of polyethylene oxide, and urethanes of polyethylene oxide and polypropylene oxide copolymers. In some embodiments, a preferred binder is carboxymethyl cellulose (CMC). Additional examples of suitable polymer binders, which may or may not be water soluble, include one or more of polypropylene (PP), amorphous polypropylene (APP), polyolefin (PL), polyethylene (PE), ethylene vinyl acetate (EVA), polystyrene (PS), polyvinyl acetate (PA), polyvinyl alcohol (PVA), polyphenylene oxide (PPO), methyl cellulose (MC), hydroxyethyl cellulose (HEC), polyacrylate, apolyacrylamide, poly(lactide-co-glycolide) (PLGA), poly(lactide) (PLA), polyglycolic acid (PGA), polyanhydrides, poly(ortho ethers), polycarprolactone, polyethylene glycol (PEG), polyurethane, polyacrylic acid, polyethylene glycol, polymethacrylic acid (PMMA), alginates, collagens, gelatins, hyaluronic acid, polyamides, polyvinylidene fluoride, polybutylene, and polyacrylonittrile.

The liquid of the fluid ceramic composition may be water miscible or immiscible and may be one or more organic or inorganic solvents or solutes. The fluid composition may contain a multiplicity of liquids. The liquid may be an aqueous liquid. For example, the liquid may be water or may be a combination of water and organic or inorganic acids or alcohols. Examples of polar organic solvents and solutes that are suitable for the liquid of the fluid ceramic composition include alcohols such as methanol, ethanol, propanol, isopropanol, and butanol, carboxyl acids, sulfonic acids, compounds containing an —OH, —SH, enol, or phenol group, formic acid, 1,4-Dioxane, tetrahydrofuran, acetone, acetonitrile, dimethylformamide, and dimethyl sulfoxide. Examples of non-polar organic solvents and solutes include hexane, benzene, toluene, diethyl ether, chloroform, ethyl acetate, and dichloromethane. Examples of inorganic solutes are hydrobromic acid, hydrochloric acid hydroiodic acid, nitric acid, sulfuric acid, perchloric acid, boric acid, carbonic acid, chloric acid, hydrofluoric acid, phosphoric acid, pyrophosphoric acid, ammonium hydroxide, alkali metal hydroxide, alkaline earth hydroxide, disodium hydrogen phosphate, ammonia, methylamine, pyridine, disodium hydrogen phosphate, disodium hydrogen phosphate dodecahydrate, disodium hydrogen phosphate heptahydrate, sodium phosphate dodecahydrate, dipotassium hydrogen phosphate, potassium phosphate tribasic, diammonium hydrogen phosphate, ammonium phosphate, trihydrate, sodium bicarbonate, NaHCO₃, NaHS, NaHSO₄, NaH₂PO₄, Na₂HPO₄, NH₄OH, NH₄H₂PO₄, (NH₄)₂HPO₄, NH₄HCO₃, and NH₄HSO₄.

The fluid ceramic composition is formed into a desired shape by any suitable method by which the desired shape may be formed. The desired shape may be any three-dimensional form. In order to make this form, the composition may be rolled, pulled, pressed, or molded to form a shape such as wire. The ceramic composition may be formed on a relatively planar surface or within a liquid, or may be cast upon an irregular non-planar template.

In many applications, it is desirable to obtain a porous ceramic article. Various embodiments of such products are useful as scaffolds for bone replacement and tissue engineering, as well as for electrodes and supports for batteries and solid oxide fuel cells, for heating elements, for chemical sensors, for solar radiation conversion, and for filters in the steel industry, among other applications. Some embodiments of the porous ceramic articles are made by replica methods, using either a positive replica or a negative replica of the ceramic article.

With the positive replica technique, a porous template, such as a sponge, is coated with a fluid ceramic composition. The ceramic composition may or may not contain additives such as binders and plasticizers that provide strength and flexibility to the coating so that it will not crack during subsequent phases of the fabrication process. Following the coating step, the coated sponge is passed through rollers to remove the excess ceramic composition and to form a thin ceramic coating over the struts of the sponge. The ceramic coated sponge is then dried and pyrolysed by heating, typically between 300° C. and 800° C., which removes fluid from the ceramic composition, removes the replica template from the ceramic composition, and solidifies the ceramic composition. Finally, if desired, the remaining ceramic coating may be densified by sintering at temperatures ranging from 1100° C. to 1700° C. depending on the nature of the ceramic material.

The positive replica technique has a disadvantage for certain indications because the struts of a ceramic article made with this technique are necessarily hollow. This results because the ceramic composition coats portions of the template that define the struts. When the template is removed, this leaves a hollow ceramic strut overlying the space where the replica strut previously existed. Also, due to the removal of the porogen strut during pyrolysis, the ceramic struts often crack during this phase of manufacture, which markedly degrades the strength of the porous ceramic article

The negative replica technique does not share these disadvantages. In this technique, a sacrificial porogen is utilized to make a template of the pores of a ceramic article, rather than of the product itself. According to this method, a negative replica of a desired porous ceramic article is made, typically by forming an assemblage of a multiplicity of discrete porogen elements, and casting a ceramic composition onto the assemblage and thereby obtaining a biphasic composition of a continuous matrix of the ceramic composition and a sacrificial phase within the matrix. The sacrificial phase may be distributed homogeneously throughout the ceramic matrix or may be assembled into a defined structure.

Following the formation of the biphasic composition, the matrix ceramic phase must be partially consolidated to form what is referred to as a “green body” or a “body” so that the porous structure of the ceramic composition does not collapse when the sacrificial porogen material is removed. Present methods of consolidation involve the use of setting agents or binders or the formation of a stiff attractive network of particles distributed throughout the matrix. Other methods include the use of sol-gel transitions based on the condensation of metal alkoxide and hydroxides in solution or by a curing process at a temperature slightly lower than that which will melt and remove the porogen materials.

The porogen materials are removed by a means that is selected based upon the nature of the porogen. Organic porogens, such as waxes, are often extracted by pyrolysis by applying long thermal treatments at temperatures between 200° C. and 600° C. Other sacrificial porogens, such as salts, ceramics, or metallic particles, are usually extracted by chemical leaching. Following the removal of the porogen, the ceramic is typically further processed, such as by kiln-firing or sintering.

Unlike the positive replica method, the negative replica method results in the formation of a ceramic article having struts that are solid, rather than hollow. Therefore, the negative replica method produces porous templates that typically have a higher compressive strength than do ceramic articles of similar porosity formed by the positive replica method.

Another advantage of the negative replica method is that it provides precise control over the architecture of the ceramic articles and can be used to produce products that are graded, either functionally or structurally. For example, gradations of pore size within a ceramic article may be obtained by grading the distribution of porogen particles of various sizes within the negative replica. In addition, gradations of composition with a ceramic article may be obtained by grading the distribution of ceramic slurry within the negative replica.

In both the positive and negative replica method, the template may be made of any material upon which a ceramic composition may be cast and which can be removed by a method that does not destroy the structure of the resulting ceramic article. Positive templates are typically made of a polymeric sponge, such as polyurethane. Other positive template materials include carbon foam and natural templates such as coral and wood. Negative template porogens include polymers such as poly(lactide) or poly(lactide-co-glycolide), salts, sugars, and waxes such as paraffin.

Certain prior art negative replica methods were tested in an attempt to make macroporous ceramic calcium phosphate (CaP) scaffolds by casting a composition onto a negative replica (i.e., a porogen) of a desired macroporous architecture of the porous article to form a body, and then removing the porogen to form the porous article. Such attempts, however, were unsuccessful for forming a sintered integrated ceramic body. It was found that the ceramic article produced in a conventional manner lacked sufficient hardness and strength, and broke into a multiplicity of pieces before and during sintering.

The presently disclosed methods are applicable to any method for forming a ceramic article, including methods as indicated above in which no template is used and those in which a template is used. If a template is used in the formation of a ceramic article, various embodiments of the presently disclosed methods are applicable to both positive and negative replica template methods.

According to some embodiments of the methods, a hardening step is performed prior to the final curing step of a ceramic article. With non-template methods of forming a ceramic article, such as when making an essentially non-porous ceramic article, the hardening step is performed before the ceramic composition has solidified and while it is still pliable. With template methods of forming a ceramic article, the hardening step is preferably performed prior to removal of the positive or negative template from the ceramic composition. Thus, with negative template methods, the hardening step is preferably performed during the formation of the green body. Because it is desirable that the ceramic composition should be as hard as possible before the template is removed, so as to minimize the occurrence of cracks in the composition, it is not preferred, although it is possible in some embodiments, to perform the hardening step described herein after the template has been removed from the ceramic composition.

In accordance with the methods of the present disclosure, the hardening step is performed by exposing the ceramic composition to a liquid extraction solvent in which non-fluid components of the ceramic composition are insoluble or practically insoluble, and in which the liquid component of the ceramic composition is miscible for a time sufficient to extract the liquid from the ceramic composition. The extraction solvent may, but does not necessarily, replace the volume of the liquid that is extracted from the ceramic composition. If the ceramic composition contains a binder, in some embodiments it is preferred that the binder is less soluble in the extraction solvent than it is in the liquid of the ceramic composition. In some embodiments, the binder is preferably insoluble in the extraction solvent.

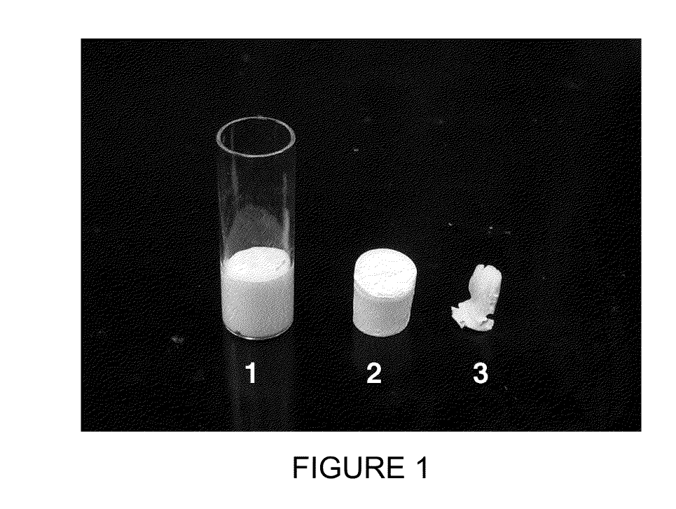

The amount of time in which the ceramic composition is exposed to the liquid extraction solvent may be varied, depending on several factors, including the materials comprising the ceramic composition, the fluid component of the ceramic composition, the liquid extraction solvent employed, and the degree of hardening that is desired. Preferably, but not necessarily, the hardening step is performed for a time sufficient that the ceramic composition will be sufficiently rigid to maintain its structural integrity in the absence of external support, for example as shown in FIG. 1. In the situation where a ceramic composition is combined with a template, the material composing the template is preferably, but not necessarily, practically insoluble or insoluble in the solvent so as not to remove the support of the template from the ceramic composition before the ceramic composition has hardened. If the template material is soluble to some extent in the solvent, then the amount of time that the template is exposed to the solvent should be adjusted so that the strength of the template is not reduced by dissolution to an extent that the ceramic composition is no longer sufficiently supported.

Extraction Solvent

The selection of the particular extraction solvent employed will depend on the identities and properties of the liquid contained within the ceramic composition and of the composition of the template, if present. For example, if the ceramic composition fluid is an aqueous fluid such as water, in some cases preferably containing a binder such as carboxymethyl cellulose (CMC), and the template is composed of paraffin, a preferred extraction solvent is some cases is a short-chain alkyl or aryl alcohol, such as methanol, ethanol, isopropanol, butanol, or phenol, or a mixture thereof. As another example, if the ceramic composition fluid is acetone, in some instances preferably containing a binder such as polymethyl methacrylate (PMMA), and the template is composed of sugar or salt, a suitable extraction solvent may be one or more of tetrahydrofuran (THF), hexane, benzene, or toluene.

Although not wishing to be bound by theory, it is postulated that the hardening of the ceramic composition that results due to the solvent extraction step of the presently disclosed methods relates to the difference in surface tension between the original liquid in the ceramic composition and the extraction solvent. For example, in the case where the original liquid in a fluid ceramic composition is aqueous, water has a relatively high surface tension compared to organic solvents, for example hexane, acetone, or alcohols such as ethanol. When a ceramic composition containing water is dried, the water exerts a force on itself and on solid components of the ceramic composition, creating stress and a tendency for the ceramic composition to crack as water is forced out by evaporation or upon heating. In contrast, replacement of water from the ceramic composition with a solvent having a lower surface tension, such as with an organic solvent, for example ethanol, acetone, or hexane, reduces the cohesive and adhesive forces of the fluid ceramic composition and results in a hardened ceramic composition with reduced stress and tendency to crack. Accordingly, when selecting an extraction solvent, it is preferred that the extraction solvent have a surface tension less than that of the original liquid of the ceramic composition.

The relative surface tensions of liquids of ceramic compositions and extraction solvents may be obtained by reference to published values for surface tensions of liquids. Alternatively, a suitable extraction solvent may be selected based on a test that reflects differences in surface tension of liquids. According to this test, equal volumes of a ceramic material are mixed in separate containers with equal volumes of two liquids, for example water and ethanol to obtain a pourable, viscous slurry. The liquid having the higher surface tension will produce a more viscous slurry than that produced with the liquid having the lower surface tension.

Another characteristic of a preferred extraction solvent is that it should be miscible in the liquid of the ceramic composition. It is also preferred in some cases that, if a binder is present in the ceramic composition, such binder should be more soluble in the liquid of the ceramic composition than in the extraction solvent. Without being limited to a particular theory, it is theorized that, when an extraction solvent is used in which the binder is less soluble than the binder is in the ceramic composition liquid, the binder will come out of solution and will function as a glue between particles of the ceramic composition and will contribute to the strength and rigidity of the ceramic composition. Thus, for example, in the case of an aqueous fluid as the liquid of a ceramic composition containing CMC in solution, extraction of water with ethanol results in increased concentration of the CMC in the liquid or a precipitation of the CMC, which causes adherence of particles of the ceramic composition.

The ceramic composition, and the template if present, are exposed to, and are preferably immersed in, the extraction solvent at a temperature below the melting point of the template. Because paraffin typically melts between 47° C. and 64° C., in certain embodiments it is in most cases preferred that, if paraffin is the material of which the template is composed, the temperature of the extraction solvent is less than 50° C. In some embodiments, the temperature of the extraction solvent is less than 47° C., and in some cases it is less than 45° C.

The concentration of the extraction solvent should be that which is sufficient to cause removal via extraction of the liquid of the ceramic composition. In some embodiments a large excess of extraction solvent is used, compared to the volume of liquid being extracted, so that the concentration of the extraction solvent is not appreciably reduced over the time period of the extraction. In some applications in which the ceramic composition liquid is water, the preferred solvent is 70% (vol/vol) ethanol. This concentration of ethanol has been found to extract water from a ceramic concentration sufficiently to increase the hardness and strength of the resulting ceramic article. If desired, a higher concentration of ethanol may be used, but care should be utilized to ensure that the ceramic composition fluid is not removed so rapidly as to crack or deform or otherwise result in structural weakness of the ceramic article.

In some embodiments it is preferred that the liquid in the ceramic composition, with or without an associated positive or negative template, is extracted by exposing the composition to sequentially higher concentrations of the extraction solvent. The stepwise increase in extraction solvent concentration is preferred in this case because a high concentration of the solvent may be utilized in this fashion which more efficiently dissolves fluid from the ceramic composition but does not dissolve the fluid as rapidly as if the ceramic composition had been exposed immediately to the higher concentration of solvent. Thus, the graded drying reduces the potential stress on the ceramic composition that would otherwise occur due to an overly rapid drying process.

For example, if the extraction solvent is ethanol, the ceramic composition, with or without an associated template, may first be exposed to the ethanol at a concentration of 70%. The ceramic composition may then be removed from the ethanol and then exposed to ethanol at a concentration of 80%. Alternatively, 95% ethanol could be added to the ethanol that the ceramic composition is in so as to raise the concentration to 80%. Following the extraction with 80% ethanol, further extraction may be performed with 90% ethanol and/or with 95% ethanol. Similar extraction procedures may be used with other combinations of ceramic composition fluid and extraction solvent.

If desired, the extraction fluid may also be utilized to remove a template, such as a sacrificial porogen utilized as a negative replica. By immersing a ceramic composition and replica template in an extraction fluid at a temperature higher than the melting point of the material of which the template is composed, the template will liquefy and will flow out of the ceramic composition and into the extraction fluid. For example, with paraffin as a template, ethanol or other alcohol may be used at a temperature above the melting point of paraffin, which is typically 50° C. or higher.

In some embodiments it is preferred that the extraction fluid utilized be one in which the material of the replica template is not soluble. In this way, the extraction fluid and the liquefied template will remain in separate phases and can readily be separated from each other. This will allow for easy collection of the template material from the extraction fluid which will allow for both the extraction fluid and the replica template material to be recycled and reused. Removal of the template material in this manner also obviates the need for pyrolysis, burning out the porogen at very high temperatures, which may potentially cause structural defects such as microcracks and therefore reduce the mechanical strength of the ceramic article.

In some embodiments the extraction of fluid from the ceramic composition is preferably performed utilizing a solvent in which a template material is not soluble at a temperature below that of the melting point of the template material and then the temperature of the extraction fluid is elevated to that above the melting point of the template material during continued fluid extraction. In this way, strengthening of the ceramic composition and removal of the template is performed in a single process.

For example, if a paraffin positive or negative replica template is utilized in the fabrication of a ceramic article, the ceramic composition associated with the template may be exposed to 70% ethyl alcohol at a temperature below the melting point of paraffin. This temperature is maintained for a sufficient time to ensure that, when the template is removed, the ceramic composition will be sufficiently strong not to collapse if the paraffin were to be removed. The temperature of the ethyl alcohol may then be increased to a temperature above the melting temperature of the paraffin, which will cause the paraffin to melt. The ethyl alcohol and paraffin may be removed and replaced with successive treatments of higher concentration ethyl alcohol for further extraction of fluid from the ceramic composition, which is now a green body.

A composition according to some embodiments of the invention is a solvent-hardened ceramic article. That is, the article was made by a process in which a liquid-containing ceramic composition is formed into a desired shape and is hardened by exposure to a solvent in which the liquid contained in the ceramic composition is soluble at a concentration and for a time sufficient to extract the liquid from the composition, and that following the extraction, the “dried” composition, which is preferably, but not necessarily completely free of liquid, is caused or permitted to solidify to form the ceramic article.

A ceramic article made by various embodiments of the above-described methods may be either non-porous or porous. If porous, it may be made by any desired method by which a porous ceramic article may be made so long as the ceramic composition is subjected to an above-described solvent extraction step prior to the final solidification of the composition to form the ceramic article. The porous ceramic article may be made with any desired degree of porosity, from 1% to over 90%. For example for calcium phosphate, as well as other ceramic articles, the porosity may be between 60% and 95%, and in some embodiments is preferably between 70% and 90%. Some embodiments of the porous ceramic articles may be made to have any desired degree of interconnectivity between pores, up to 100% interconnectivity. For example, in some embodiments, interconnectivity between pores is in the range of about 70-99%. The porous ceramic article may be made by a negative replica method in which discrete porogen particles are used to define a template upon which a ceramic composition is cast. One potential advantage of embodiments that use the negative replica method is that the interconnectivity of the pores of the product may be controlled by heating or otherwise causing individual elements of the sacrificial porogen to coalesce to a desired degree which will correspond to the degree of interconnectivity of pores in the final ceramic article. Another potential advantage of embodiments that employ the negative replica method is that a resulting solvent-hardened ceramic article may be a porous article having uniformity of distribution of pores, pore sizes, and composition or any of these characteristics of the article may be varied to provide a porous article that varies spatially in the distribution of pores, of pore sizes, and/or of composition. In some embodiments, non-porous articles may also be compositionally graded.

The resulting ceramic articles have a variety of different uses. The increased compressive strength of various embodiments of the ceramic articles are of use in many fields, including, but not limited to, for making biodegradable ceramic articles for implantation into the body of humans and other animals as well as for structural materials for buildings and electronics, among others.

In some embodiments, a particular use of a ceramic article disclosed herein is for implantation in order to repair bone. Synthetic biodegradable ceramic bone graft materials made by conventional methods of manufacture have compressive strength less than that of bone. Additionally, the ceramic bone graft materials typically lose a significant portion of their initial strength over time as the synthetic bone is absorbed into the body. Various embodiments of the methods disclosed herein, when utilized for strengthening biodegradable ceramic bone grafts, will potentially provide a significant contribution to this field.

The presently disclosed methods provide for the controlled formation of macroporous regions that are highly interconnected. At least 70% of the pores in a ceramic article are interconnected, and in many embodiments interconnectivity is up to about 100%. In various embodiments, a ceramic article is designed to have about 70, 75, 80, 85, 90, 95 or 99% interconnectivity, for example. Greater porosity results in greater strength, and thus CaP scaffolds fabricated using the disclosed methods can be used to facilitate healing and repair of compact or cortical bone, including repairs to large bone defects or injury, including craniofacial defects. The presently disclosed methods can also produce pores of a predetermined size that are highly interconnected and more likely to allow bone ingrowth, becoming filled with newly formed bone and bone marrow cells easily. Furthermore, by controlling pores size and formation of a gradient of zones containing pores of increasing or decreasing size, the present methods provide a method of generating a functionally graded scaffold that mimics the gradient of natural bone, its strength and other characteristics. The high degree of interconnectivity of the pores that can be achieved using the present method allows for the fabrication of excellent mimics of trabecular (spongy or cancellus bone). Thus with only minor adjustments, the presently disclosed method provides ceramic articles that can be used as synthetic bone to repair both types of bone.

Ceramic Articles with Multiple Architecture Zones

Some embodiments of the ceramic articles produced as described herein include graded CaP ceramic scaffolds, containing multiple zones providing various advantages. For example in one embodiment, a two-zone graded CaP ceramic scaffold comprising an outer zone of dense pore-less ceramic and an inner zone of a porous scaffold is designed to mimic naturally occurring bone having an inner zone of cancellous bone and an outer zone of cortical bone. In another embodiment, a three-zone graded CaP ceramic scaffold is contemplated that comprises three-zones, a central porous cylinder, a middle cylinder of increasing density and a peripheral cylinder. The presence of a central porous cylinder may be used to delivering growth factors and/or cells that may enhance osteointegration. Alternatively, in some embodiments a central porous channel is provided to facilitate attachment of hardware during surgery, as, for example, when using screws, intramedullary nails and inserts as well as other devices. Likewise, in some embodiments, a middle cylinder of denser ceramic is present to provide high compression strength, comparable to human bone.

Furthermore, some embodiments of the ceramic articles that are produced as described herein may also incorporate biopolymers such as, but not limited to, chitosan, polylactic acid or polylactide (PLA) polyglycolide (PGA), poly(lactic-co-glycolic acid) (PLGA), hyaluronic acid, hyaluronate salts, hydroxypropylmethyl cellulose, dextran, alginate, agarose, polyethylene glycols (PEG), polyhydroxyethylenemethacrylats (HEMA), synthetic and natural proteins, or collagen. The incorporation of biopolymers may improve the torsion and bending strength of the composite scaffolds.

Thus, the technologies disclosed herein provide unique bone graft methods and fabrication techniques. These techniques allow control of gradual and spatial change chemistry, porosity, and thus the structure across a bone graft. This facilitates seamless integration of different materials and properties, including, but not limited to, increased torsion and bending strength due to incorporation of polymers into the already strong, with regards to compression strength, ceramic articles produced by the methods of the present disclosure. Various embodiments of these methodologies provide novel and improved methods of generating materials for use in bone grafts for the repair of large load-bearing bone defects.

The methods disclosed herein are useful in the creation of macroporous structures which have a high degree of interconnectivity between pores and a high compressive strength. Exemplary methods produce a sintered macroporous CaP ceramic article by a negative replica method, which articles may have about 70-100% interconnectivity between pores, a porosity up to or even higher than 70%, and solid struts between pores. The inventors have found that similar articles produced by prior art negative replica methods lacking the solvent extraction step were not sufficiently strong to withstand sintering temperatures used to solidify the ceramic article. It is believed that no macroporous article made by negative replica methods and having the above-described high interconnectivity between pores has been produced prior to the presently disclosed methods.

The negative template-casting method disclosed herein provides for fine control of macroporous structures by varying the sizes of beads utilized and their arrangement. For instance, scaffolds with two ranges of pore sizes, 600-800 μm and 350-500 μm, were successfully fabricated (Group I and Group II, respectively, in Table 1). High interconnectivity of pores was also readily achieved in these scaffolds regardless of pore size. Analysis using scanning electron microscopy (SEM) revealed reticular structure of the scaffolds in which each and every macropore interconnects to multiple neighboring pores. These interconnective windows were at the macroscale, averaging 330±50 and 440±57 μm, respectively, dependant on the sizes of paraffin beads. Table 1 describes the physical characteristics of two scaffolds of different porosity fabricated by otherwise identical negative template-casting method that includes an above-described solvent extraction step.

In some embodiments, in which the ceramic article comprises two, three, or more distinct zones, at least two of the zones are interconnected.

TABLE 1

| Samples | Group 1 | Group 2 |
|---|---|---|
| Macro pore sizes/μm | 600-800 | 350-500 |
| Interconnective opening sizes/μm | 440 ± 57 | 330 ± 50 |
| Strut thickness/μm | 220 ± 90 | 140 ± 84 |
| Micro pore sizes (in struts)/μm | 1.2 ± 0.3 | 1.1 ± 0.4 |
| Grain sizes/μm | 1.5 ± 0.4 | 1.6 ± 0.4 |
| Apparent density g/cm³ | 0.66 ± 0.06 | 0.63 ± 0.03 |
| Total porosity*/Vol % | 79 ± 1 | 80 ± 2 |
| Linear shrinkage rate/% | 50 ± 1 | 50 ± 1 |

*Porosity was estimated by dividing the apparent density by theoretical density of β-TCP (3.156 g/cm³)

In some embodiments, various porosities of scaffolds, such as, but not limited to, between about 70% to about 90% can readily be obtained by controlling the template process which is determined by paraffin bead size and arrangement. In some embodiments, the porosity is lower than 70% or higher than 90%. In addition to macroporosity, microporous structures on struts were also achieved by template-casting method, which may potentially improve the scaffold performance in vivo.

In making one embodiment of the macroporous scaffold, a multiplicity of particles, such as beads, are arranged to form a negative replica. Typically, but not necessarily, the particles are arranged within a container, such as a tube. The particles are caused to agglomerate, such as by heating the particles to a temperature at which they begin to melt and become tacky, causing adjacent particles to adhere to each other, and thereby forming a unitary mold structure. A ceramic composition, such as a CaP ceramic composition, is then introduced into the container to fill the spaces not occupied by the negative replica.

The porosity of the scaffold may be controlled in various ways. Because the template is a negative replica, the use of larger size particles will provide a template of greater porosity than will be obtained using particles of smaller size. Additionally, increased melting of the particles, such as by increasing the temperature and/or time of heating, will result in increased surface of adherence of one particle to another, thereby resulting in increased porosity.

In some applications, a multiplicity of containers are situated one within another so as to form a multiplicity of zones. Within the different zones, particles of different sizes or shapes may be utilized in order to vary the architecture, such as the porosity, of the mold structure within each zone. Within the different zones, different ceramic compositions may be introduced so as to vary the composition of the scaffold from zone to zone.

After the ceramic composition is introduced into the container, the ceramic compositions are exposed to a solvent, as described above, to harden the ceramic compositions and remove liquid that is contained within the compositions. The negative replica is removed, such as by chemical or heat treatment, and the scaffold is permitted to solidify, such as by air drying or sintering.

In some applications, the resulting scaffold is loaded with cells, such as mesenchymal or other stem cells, or with a growth factor, such as bone morphogenic protein (BMP) or an angiogenic growth factor such as vascular endothelial growth factor (VEGF) or transforming growth factor (TGF). The scaffold may also be loaded with a pharmaceutically active agent, such as an antibiotic or an analgesic.

In some cases, an above-described scaffold is coated or infiltrated with a material such as chitosan or other polymer. The coating may facilitate the incorporation of cells, drugs, or growth factor onto the scaffold. If the scaffold is to be coated, the coating is typically applied before loading the scaffold with the cells, drugs, or growth factors. For some applications, bending strength of composite scaffolds is increased as a result of a polymer coating on a porous ceramic.

Coating and/or loading the scaffold may be accomplished by any suitable means that provide for coating or loading CaP scaffolds. For example, coating and loading may include spraying, painting, or dropping the coating material or a liquid containing the loaded material onto the scaffold, or by immersing the scaffold in such a liquid. The immersion method is preferred in most cases because the inventor has found that this method provides for more precise regulation of loading and elution based on pore size.

In various embodiments, the CaP scaffold may have zones of different architectures, which can be used to control biodegradation, spatial and or temporal, of the implanted scaffold. This permits a temporally and spatially controlled osteogenesis. In a preferred embodiment, the architecture of the scaffold is arranged to form a biomimetic scaffold that resembles the architecture of natural bone. According to this embodiment, a macroporous scaffold is made having a multiplicity of zones, such as an inner zone and an outer zone. The inner zone has a higher porosity than that of the outer zone. In this way, the inner zone mimics the architecture of cancellous bone and the outer zone mimics the architecture of cortical bone.

In some embodiments, multi-zone scaffolds are constructed such that the regions mimic natural bone and appropriate zones of the porous ceramic network are infiltrated biopolymer (such as but not limited to, nano-hydroxyapatite or chitosan) to form integrated composites. Scaffolds constructed using the disclosed methods can also incorporate open regions (holes) through which, for example, nerve or vascular tissue may be passed, thus facilitating the use of the present scaffolds in repair of spinal bone.

In some embodiments, a CaP scaffold produced as described herein is used to repair bone defects. For repair of bone defects, the scaffold may or may not be loaded with a growth factor, such as BMP. In exemplary embodiments, the CaP scaffold has been utilized in long bones of a rabbit. Repair of bone defects in the rabbit was obtained utilizing either BMP loaded CaP scaffolds or CaP scaffolds without BMP loading. Repair was more rapid, however, with scaffolds that were loaded with BMP.

To further illustrate the above embodiments, the following examples are provided. It is to be understood that these examples are provided for illustrative purposes and are not to be construed as limiting the scope of the claims.

### EXAMPLES

### Example 1

### Forming a Ceramic Article

A. In general, exemplary ceramic articles were fabricated as follows: Paraffin beads were prepared by a conventional water-suspension method. The paraffin beads were sifted in order to obtain beads with diameters ranging from 1.2 to 1.8 mm. The sifted beads were filled into polyethylene cylinder tubes. The filled tubes were placed into warm water at a temperature of about 50° C. to allow the beads to soften and to coalesce into a unitary mold structure.

A fine tricalcium phosphate (TCP) powder was mixed with distilled water at various weight ratios of 1:(0.2-10). This mixture was stirred and carboxymethyl cellulose (CMC) was added at various weight ratios of 1:(20-1). The mixtures were stirred until a homogenous slurry was obtained.

The slurry was poured onto the top of the paraffin mold. The mold with the slurry was placed into a vacuum chamber for at least 10 minutes, at which time the chamber was filled with air and the paraffin mold was checked to determine if it had been completely filled with the slurry. If not completely filled, additional repetitions of the pouring of the slurry onto the mold and the exposure to the vacuum were performed until it was determined that the paraffin mold was completely cast with the slurry to make porous ceramic bodies for making macroporous ceramic articles.

Another set of samples was prepared by directly filling the slurry into the polyethylene cylinder tubes, without prior filling of the tubes with paraffin beads. These molds were therefore not cast upon a negative template and resulted in solid non-porous ceramic articles, sometimes referred to herein as “scaffolds.”

The ceramic bodies, porous and non-porous, were soaked in 70% ethyl alcohol at a temperature between 30° C. and 60° C. for at least 30 minutes. The temperature was then increased to between 60° C. and less than 100° C. and maintained for no less than 30 minutes in order to melt and remove the paraffin molds. The alcohol and melted paraffin were replaced with 80% to 95% ethyl alcohol at 60° C. to less than 100° C. and maintained for at least 30 minutes. The ethyl alcohol was replaced with new ethyl alcohol at the same concentration and maintained for at least 30 minutes. A control group for each of the solid and porous ceramic bodies was air dried, rather than applying this solvent-based solidifying and drying fluid extraction process. All samples were then placed into an electric furnace and were heated to a temperature of 1100° C. to 1300° C. for a period of 3 hours to produce sintered porous and non-porous ceramic articles.

B. Fabrication of a calcium phosphate scaffold with zones of differing porosity.

One specific embodiment of the present methods to fabricate a calcium phosphate scaffold involves the following steps. The paraffin beads used to form pores in the scaffold were prepared prior to the construction of the molds. Paraffin beads were formed using a water quenching technique in which one liter of water was heated on a hot plate and while maintaining the temperature between 75-80° C., 150 g paraffin wax and 5 g carboxymethylcellulose (CMC) (3.3% by wt of the wax) were mixed in with continuous stirring until the wax and CMC had completely dissolved. The water is allowed to cool slightly and when it has reached about 65-75° C., the solution appeared slightly creamy but not translucent. The rate of stirring was increased slightly and the mixture was then very quickly quenched by adding approximately 1 liter of ice water. The beaker was promptly removed from the hot plate and the water and wax beads were poured through the sieves and a series of cold water rinses were applied over the beads within the sieves. These bead construction steps were repeated until sufficient batches of beads had been made. Once a sufficient number of beads were made, they were dried under the hood (with the light on, vacuum on high) for at least 48 hours. Once the beads were dry, they were sifted to collect those having the desired size. Large pores often are formed using dried bead diameters of 1.18 to 1.70 mm and small pores are formed by using dried beads with diameters between 0.71 and 1.00 mm.

The molds for the scaffolds were constructed from 24 well culture plates. The sides of the well plates were removed and holes punched between the wells to allow water to fully contact the sides of the plate without actually entering the wells themselves. These molds were then coated in wax, filled with beads, and partially melted to ensure stability.

A bowl of melted paraffin wax was maintained at or above 53° C. (the melting point of the paraffin) oven for coating the plates. The desired number of well plates were coated in paraffin wax by dipping the plates for approximately 3 seconds, correct side up. The plates were then inverted and allowed to cool on a paper towel. To avoid the pooling of excess wax and obstruction of the well opening, the plate was moved from its original spot on the towel after about 10 seconds. The desired wax coating was thick enough to make the wells almost completely opaque. If the wells appeared translucent, they received a thicker coating of wax.

A water bath preheated to 53° C. with a water level adjusted such that it just touches the top of weighted plates was prepared. Once the desired architecture for the mold was determined, each well was filled with the desired size of beads. If the structure was to be graded, a straw or aluminum-coated stick was used to set the beads in the desired locations and gentle pressure was then applied, such that the beads did not move when the well plate was inverted. After all of the wells had been filled with the desired beads, the mold plates were placed in the water bath for 30 minutes while being careful to avoid contact between the water and the wax beads. The mold plates were then removed and dried overnight in a hood to facilitate the evaporation of all the excess water.

An important factor for scaffold properties is the formulation of the slurry. In order to create a TCP slurry with a slurry to water ratio of 1:2.5, the following formulation was applied. Forty (40) mL of DI water was placed in a 100 mL beaker with the largest possible stir bar and preheated on a hotplate to between 7° C. and 80° C. To this water 12g of fine TCP powder (Nanosize β-TCP, Nanocerox, Inc., Ann Arbor, Mich.) Paraffin beads will be prepared by conventional water-suspending methods and classified into different diameters using a series of sieves. was added while maintaining a vortex in the slurry. Once the TCP was a uniform slurry, 18 drops (measured with FISHER brand pipettes, cat #13-711-9am (Fisher Scientific, Pittsburgh, Pa.) of Antifoam A, polydimethylsiloxane (Spectrum, A132) and 36 drops (measured with FISHER brand pipettes) of ammonium dispersing agent, ammonium polyacrylate (APA) (Darvan, 821A) were added. Then add 1.2 g of magnesium acetate (MgAc) was added and the slurry was allowed to mix for 30 minutes at a medium spin (a small vortex was always present). After the 30 minutes had passed, 2.4 g of CMC (Fisher Scientific) was added, very slowly and in small aliquots. It was best to allow each aliquot of CMC to dissolve before adding the next aliquot.

Once all the CMC had dissolved into the slurry it was allowed to stir for 1 hour. Before the hour expired, however, the weight of the slurry was verified to confirm the water content. The beaker weight+stir bar (both totaled approximately 70 g)+slurry (18 g)+water (18*2.5)=total weight in g of about 133.0 g. Water was added if necessary, or if there was an excess of weight water was removed by evaporation by allowing the mixture to remain on the heat. Once the slurry had achieved the correct weight, the stir bar was removed and the beaker sealed using plastic wrap and a rubber band. The plastic wrap was labeled and covered in foil, and allowed to cool to room temperature.

Once the slurry had cooled below 53° C. and the cast molds had dried, the mold wells were filled with the viscous slurry. A syringe was used to top off each well with slurry until a slight crown of slurry rose above the wall of the mold. To assure complete filling of space within the mold, the molds were placed within a desiccator and a vacuum applied for between 1 to 3 minutes. The slurry was carefully observed and began to bubble rapidly, at which point the vacuum was released by allowing air to re-enter the desiccator before the bubbles spilled into adjacent wells. If any of the wax layers within the wells was still visible, additional slurry was added to wells. Then filling process was repeated multiple times until all wells were filled. Slurry was considered fully infiltrated when the slurry level no longer changes between vacuum applications. After the vacuum process was complete, excess slurry outside the wells was removed using suction.

Filled plates are immediately submersed into (0.5 L/plate) preheated 70% ethanol (Fisher Scientific) at approximately 30-40° C. The plates were placed, tilted off vertical, in the warmed alcohol where they remained for a minimum of 48 hours. After which, the well plates were moved to another container containing 70% ethanol at 30-40° C. The temperature of this 70% ethanol bath was increased to approximately 70-80° C. and maintained for 2 hrs. The well plates were then removed and the green bodies were demolded by quickly inverting the well plates over a wire mesh. While removing the plates they were held in a vertical position to avoid drawing up melted wax. The mesh, on which the green body scaffolds now lie, was immersed in another container of 70% ethanol warmed to 70-80° C. for 2 hours. The green body scaffolds were then transferred to a container of 90% ethanol warmed to 70-80° C. for 2 hours and finally a container of 95% ethanol warmed to 70-80° C. for 2 hours. The scaffolds were removed and allowed to dry for at least 2 hours prior to firing.

Firing was done in a high temperature furnace used to heat the ceramics to 1250° C. Scaffold disks were placed in alumina dishes. The scaffold disks were separated such that they don't touch the walls or each other. The alumina dishes were stacked inside with the lids only partially covering them. The furnace cycled up from room temperature to 1250° C. at the rate of 5° C. per minute. It remained at 1250° C. for 3 hours and then reduced temperature at the rate of 5° C. per minute back down to shut down (room temperature). Once the firing had been completed (about 12 hours) the dishes were removed and allowed to cool. In order to make precise final adjustments to the shape, weight, specific dimensions, etc. of the scaffolds, sandpaper was used to polish the scaffold disks to the desired form. In this manner, controlled formation of macroporous regions that are highly interconnected (at least 70% and, in some cases, about 100% interconnectivity between adjacent pores), and creation of pores of a predetermined size or formation of a gradient of zones containing pores of increasing or decreasing size is accomplished. For some applications, a functionally graded scaffold that mimics the gradient of natural bone, its strength and other characteristics is formed. The high degree of interconnectivity of the pores that can be achieved using the present method allows for the fabrication of excellent mimics of trabecular (spongy or cancellus bone). Thus with only minor variations of this method custom designed ceramic articles may be prepared for use as synthetic bone to repair both cancellous and cortical types of bone.

### Example 2

### Testing of the Ceramic Articles

The porosity of the porous ceramic scaffolds of Example 1A-B was calculated by dividing the apparent density of the scaffold with the TCP theoretical density of 3.14 g/cm3 and was determined to be about 73%. The apparent density of the scaffolds were determined by measuring the mass of the scaffold and dividing by the volume of the scaffold.

Macromorphology and three-dimensional structure of the scaffolds were determined by micro computed tomography (micro CT). Scanning electron microscopy was used to determine the microstructure of the scaffolds. Maximum compressive strength of the ceramic articles prepared in Example 1A-B was determined by using a mechanical tester (INSTRON 4465, Instron Corp., Canton, Mass.). The maximum compressive strength was measured and, for a macroporous scaffold made with the solvent extraction step, having approximately 100% connectivity and having pore sizes of 350-500 μm or 600-800 μm, was determined to be 17+/−4 MPa. It was not possible to determine the compressive strength of the similar macroporous scaffold made without the solvent extraction step, because these scaffolds invariably cracked into pieces prior to or during the exposure to sintering temperatures.

A plastic tube filled with a slurry of a ceramic composition prior to drying is shown on the left side of FIG. 1. In the middle of FIG. 1 is shown a macroporous green body dried by the solvent-extraction method described herein and on the right side of FIG. 1, a green body dried by exposure to air at room temperature. As shown in the middle of FIG. 1, the solvent extraction drying step maintained the integrity of the green body whereas, as shown in the right side of FIG. 1, air drying did not maintain the integrity of the green body, which crumbled and cracked into a multiplicity of pieces.

Similarly, maximum compressive strength of a dense non-porous article made with the solvent extraction process of Example 1A-B was determined to be 297.8+/−73.0 MPa. The comparable dense non-porous articles made without the hardening step disclosed herein invariably developed cracks during sintering and so were not tested for compressive strength.

These results demonstrate that both porous or non-porous ceramic articles (scaffolds) may be made by the method of the present disclosure and that such ceramic articles are able to withstand processes such as sintering. Moreover, they show that articles made by the method of the present disclosure have a very high compressive strength.

### Example 3

### Comparison of Strength of Macroporous Scaffolds

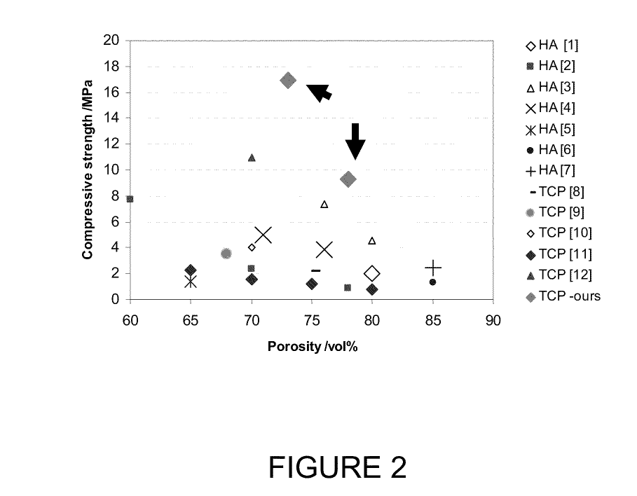

The compressive strength of additional macroporous CaP scaffolds made according to the method of Example 1A-B and having a porosity of 73% was tested by the method of Example 2 and determined to be 16.86 MPa+/−3.60 MPa. This was compared to the strength of prior art macroporous scaffolds made with various methods as reported in the scientific literature. See, Hing, Best, and Bonfield, ibid.; Liu, ibid.; Sepulveda, et. al, ibid.; Ramay and Zhang, ibid.; Almirall, et al., ibid.; Cyster, et al., ibid.; Silva, et al., ibid.; Uemura et al., ibid.; Sous, et al., ibid.; Guo et al., ibid.; Kwon, et al., ibid.; Milosevski, et al., ibid. The results are shown in FIG. 2, which is a graph plotting compressive strength in MPa on the Y-axis and porosity in volume % on the X-axis.

As shown in FIG. 2, the maximum compressive strength of the macroporous scaffold made according to a method described herein (indicated by the arrow) is markedly higher than is that of scaffolds constructed using different methods described by others. This is true even when the scaffolds made according to the methods of others had a lower porosity which, because of higher mass per volume, would have been expected to be stronger than higher porosity scaffolds constructed using the present methods.

### Example 4

### Compressive Strength of Cortical Bone and Biomimetic CaP Scaffold

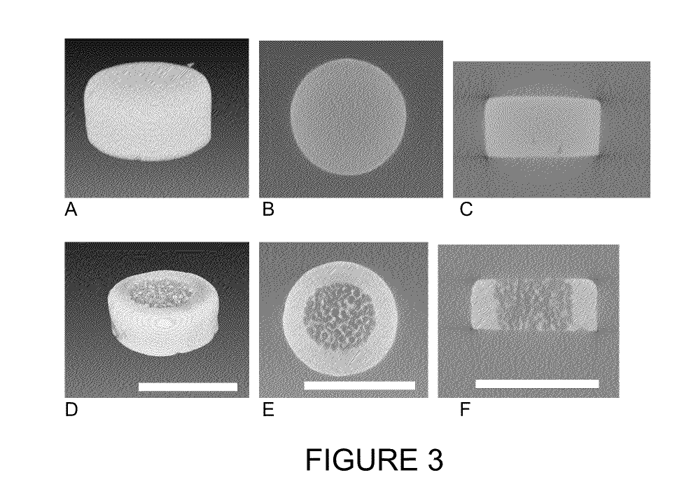

A dense CaP ceramic article, referred to in this example as a scaffold even though the article lacks pores, was made according to Example 1A. FIG. 3A-C shows a 3-dimensional and two 2-dimensional Micro CT images of dense scaffold showing the lack of pores. This pore-less scaffold was made to mimic the structure of cortical bone.

A graded CaP ceramic scaffold, containing an outer zone of dense pore-less ceramic and an inner zone of a porous scaffold, was made according to the method described in Example 1A-B. FIG. 3D-F is a 3-dimensional and two 2-dimensional MicroCT images of the scaffold showing the two-zone graded ceramic scaffold having 600 μm to 800 μm pores in the inner zone and lacking pores in the outer zone. This two-zone scaffold was made to mimic naturally occurring bone having an inner zone of cancellous bone and an outer zone of cortical bone. The two-zone graded ceramic scaffold was made by filling a tube with paraffin beads followed by filling of the tube with a ceramic slurry and filling an outer concentric tube with the slurry without first filling this outer tube with the beads.

The maximum compressive strength of the dense ceramic scaffold and the two-zone ceramic scaffold was determined as described in Example 2 and was compared to the strength of cortical bone reported in An Y H and Draughn, R A, “Mechanical Testing of Bone and the Bone-Implant Interface”, CRC Press, Boca Raton, Fla. (2000). The strength of cortical bone reported in An and Draughn is 200+/−36 MPa (from 133 to 295 MPa). The strength of the non-porous dense CaP scaffold was determined to be 297.8+/−73.0 MPa. The strength of the two-zone scaffold, mimicking the structure of bone having both cortical and cancellous zones, was determined to be 153.9+/−2 9.2 MPa.

The results of this study were surprising because, not only was the compressive strength of the dense scaffold substantially higher than that of cortical bone, the two-zone scaffold also had a compressive strength similar to or somewhat higher than that of cortical bone. It is to be noted that the compressive strength of bone having both cortical and cancellous portions will naturally be less than that of cortical bone alone. Therefore, the data establish that at least some embodiments of the CaP scaffold have a strength that is equal to or higher than that of natural bone. Many embodiments of the scaffolds disclosed herein are expected to be able to withstand functional loading when used as implants for long bone grafting.

### Example 5

### Manufacture of Macroporous Scaffold

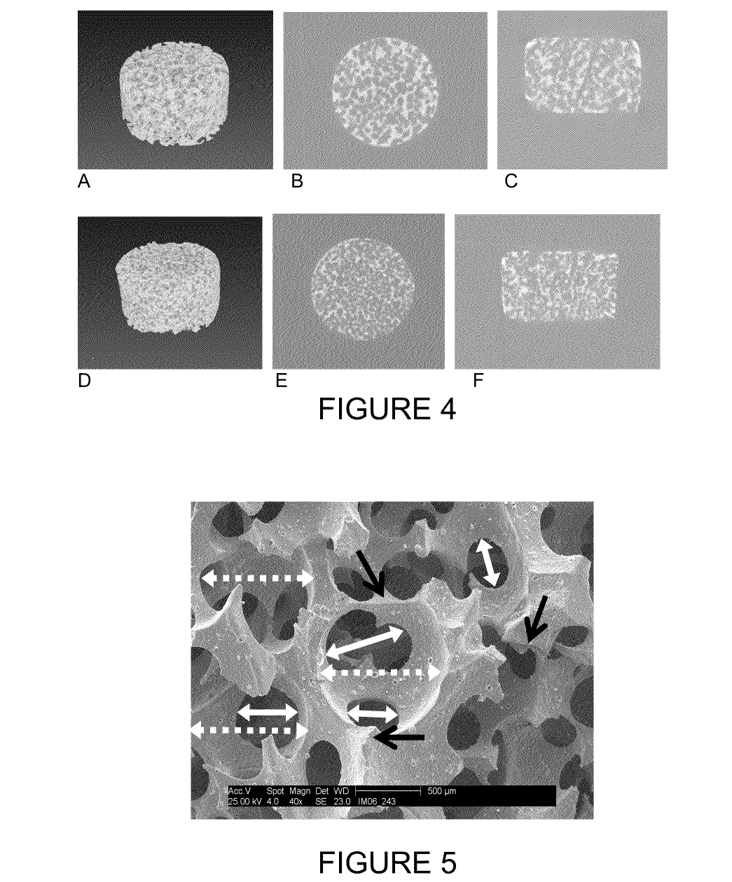

Macroporous scaffolds were made according to Example 1A to produce scaffolds having pores between 600 μm to 800 μm, shown in FIG. 4A-C, and between 350 μm and 500 μm, shown in FIG. 4D-F.

### Example 6

### Interconnection of Pores of Macroporous Scaffold

A macroporous scaffold having pores between 600 μm to 800 μm was made according to Example 1A-B and was imaged by scanning electron microscopy, as shown in FIG. 5. The interconnective pore size was determined to be 440+/−57 μm. The struts between pores (indicated by black arrows) are solid due to formation of the scaffold by the negative replica method. Numerous pores are indicated by dashed white arrows and interconnective pores which fluidly connect adjacent pores to each other are indicated by solid white arrows. The interconnectivity and interconnected pores of scaffolds are important for bone regeneration. It is these interconnected pores, not separated pores, that allow blood vessel ingrowth and sustain the regenerated bone tissues. The term “interconnectivity” refers to the number of open pores relative to all pores, including open pores and closed pores, in a ceramic article. The pore size and percent of interconnected pores may be readily manipulated using a disclosed template-casting method. In addition, the surface morphology of the scaffolds, either nanoporous or having a dense feature, may also be readily manipulated using these methods. The ability to vary surface morphology as desired allows the user to regulate drug loading and to change the drug kinetics for treatment at a bone defect site.

### Example 7

### Manufacture of Radially Graded Macroporous Scaffold

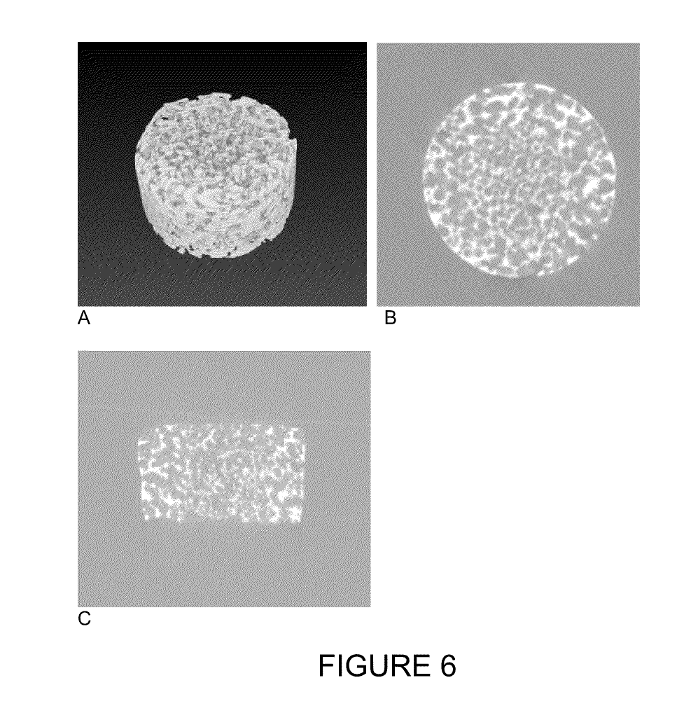

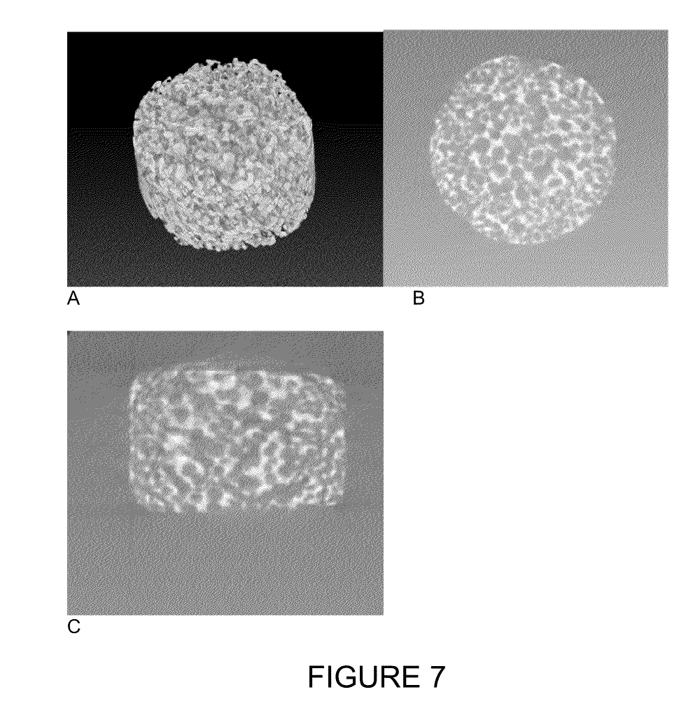

Macroporous scaffolds were made according to Example 1A-B except that two concentric polyethylene tubes were utilized and paraffin beads of two different sizes were respectively filled into each of the tubes. FIG. 6A-C shows a 3-D and two 2-D Micro CT images of a radially graded porous ceramic article (scaffold) in which an inner zone of the article contains pores between 350 μm to 500 μm in diameter and an outer zone contains pores between 600 μm and 800 μm. FIG. 7A-C shows a 3-D and two 2-D Micro CT images of a radially graded porous ceramic article in which an inner zone of the article contains pores between 600 μm and 800 μm in diameter and an outer zone contains pores between 350 μm to 500 μm in diameter.

### Example 8

### Manufacture of Vertically Graded Macroporous Scaffold

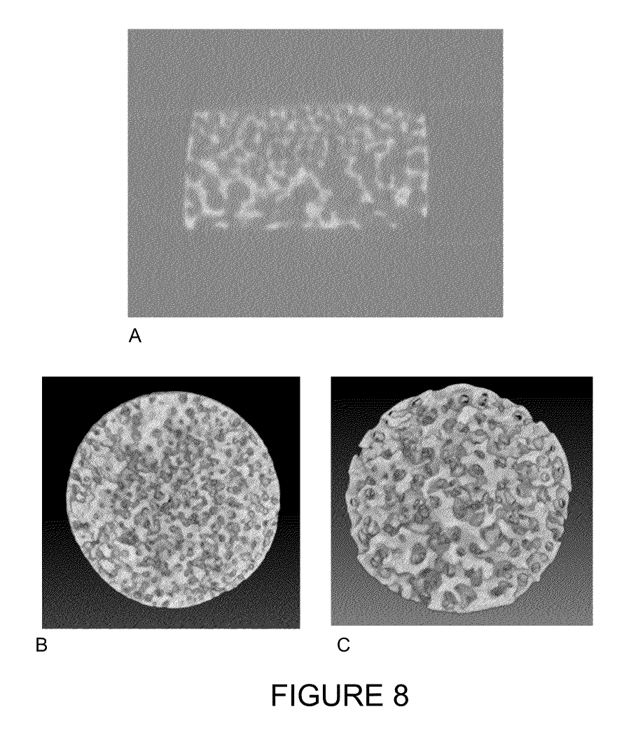

A macroporous scaffold was made according to Example 1A-B except that two differently sized populations of paraffin beads were sequentially used to fill the polyethylene tube. FIG. 8A-C shows a 2-dimensional Micro CT image of the resultant vertically graded macroporous structure in which the top portion has smaller pores of 300 μm to 400 μm and the bottom portion has larger pores of 600 μm to 700 μm, a top view 3-dimensional Micro CT image of the vertically graded macroporous structure showing the smaller pores at the top surface, and a bottom view 3-dimensional Micro CT image of the structure showing the larger pores at the bottom surface.

### Example 9

### Manufacture of Compositionally Graded Macroporous Scaffold

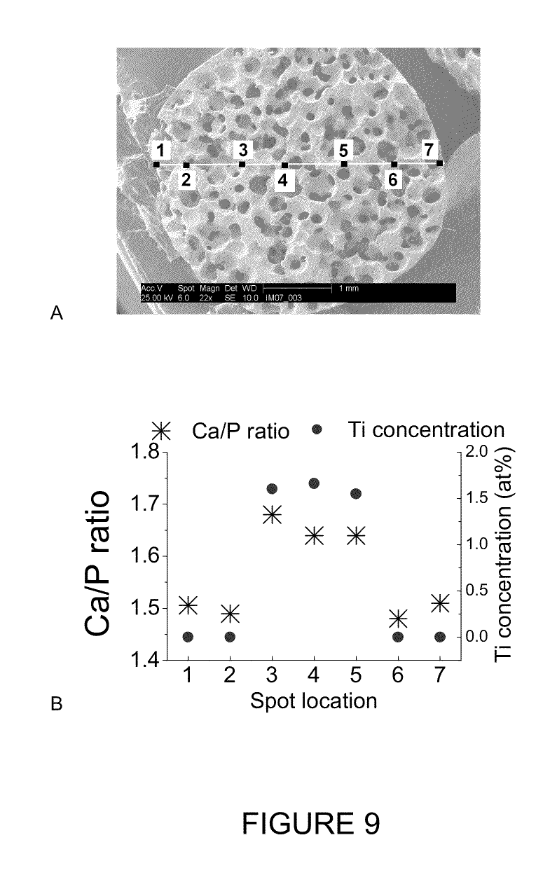

A macroporous scaffold was made according to Example 1A-B except that two concentrically arranged polyethylene tubes were utilized and different compositions of ceramic material were poured into each tube. The centrally positioned tube contained a ceramic material that was relatively hydroxyapatite (HA) enriched, had a calcium/phosphorus (Ca/P) ratio of about 1.64-1.68:1, and contained titanium oxide. The peripherally positioned tube contained a ceramic material that was relatively tricalcium phosphate (TCP) enriched, had a Ca/P ratio of about 1.48-1.51:1, and did not contain titanium oxide. FIG. 9a shows measurements obtained at selected locations in the scaffold. FIG. 9b shows the varying composition of the scaffold at each of these selected locations.

As shown in FIG. 9b, the Ca/P ratio was higher, between 1.64-1.68:1, in the central HA enriched area of the scaffold compared to the Ca/P ratio in the peripheral area of the scaffold which were between 1.48-1.51:1 in the peripheral areas of the scaffold. Additionally, higher concentrations of titanium, 1.55-1.66:1, were present in the central area and the amount of titanium in the peripheral areas was at or about zero. This result established that there was little movement of slurry components during the template-casting procedure and that this and similar methods may be used to produce compositionally graded ceramic articles.

### Example 10

### Controlled Degradation of CaP Scaffolds

Four groups of CaP (β-TCP) scaffolds were made according to Examples 1A-B and 7 above to produce (Group A) scaffolds with uniform large pores (between 600 μm and 800 μm), (Group B) scaffolds with uniform small pores (between 300 μm to 400 μm), (Group C) radially-graded scaffolds with central small pores and peripheral large pores, and (Group D) radially-graded scaffolds with central large pores and peripheral small pores. Each of the four groups of scaffolds had the same porosity, between 70-73%.

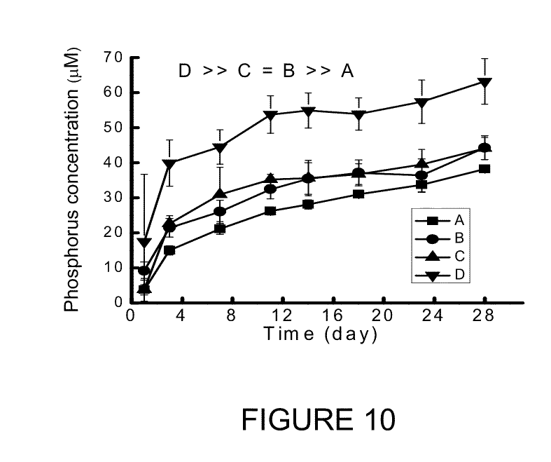

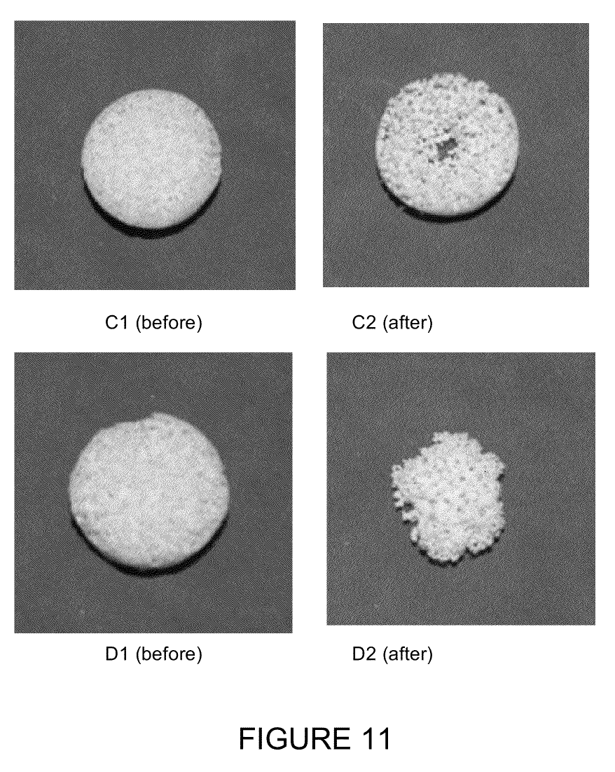

The scaffolds were soaked in Tris buffer (pH 7.4) at 37° C. The dissolution rates of the four groups of scaffolds were measured for a period of 4 weeks. Data is shown in FIG. 10, in which the graded CaP scaffolds with central large pores and peripheral small pores (Group D) exhibit significantly greater dissolution rate than those with uniform small pores (Group B) and the other graded scaffolds with central small pores and peripheral large pores (Group C) in the course of dissolution. In addition, the scaffolds with uniform large pores had the lowest dissolution rate of all groups. No significant difference in dissolution rate was noted between the scaffolds with uniform small pores and the graded scaffolds with central small pores and peripheral large pores. It is postulated that the greater dissolution rate of the scaffolds with uniform small pores is due to their higher surface area compared to those with uniform large pores. It is also postulated that a tension stress caused by the graded architecture resulted in an increased dissolution rate for the graded scaffolds of Groups C, those with central large pores and peripheral small pores. Scaffolds of Group C and Group D were immersed in acidic buffer media (pH 3). The degradation pattern of these scaffolds is shown in FIG. 11. The scaffold regions with the greatest dissolution rate were observed to be the regions with smaller pores regardless of the location of the regions.

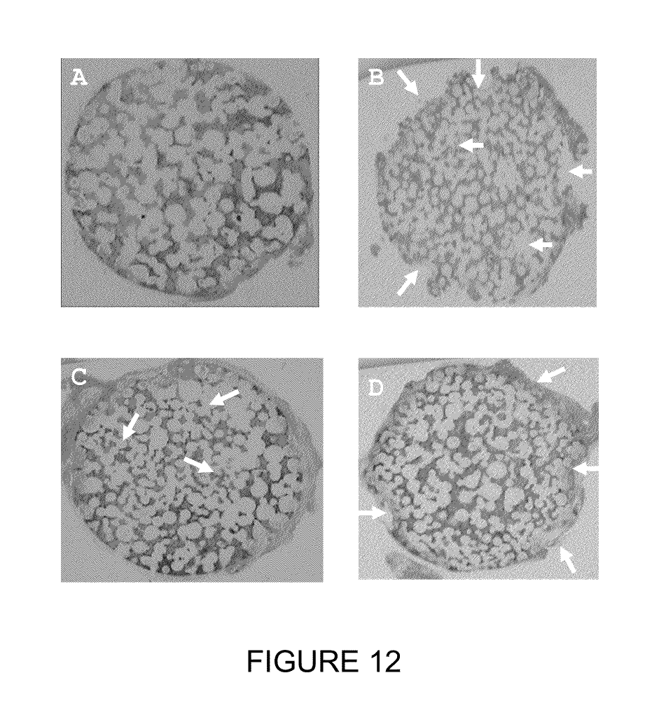

The in vivo biodegradation of the scaffolds was also evaluated. Scaffolds were implanted subcutaneously into mice and the morphology changes were evaluated using non-decalcified histological samples. The results, shown in FIG. 12, were similar to the in vitro study above. FIG. 12, panels A-D, show 4 different CaP scaffolds one month after implantation. Panel A is a scaffold from Group A with uniform large pores of 600 to 800 μm. Panel B is a scaffold from Group B with uniform small pores of 350 to 500 μm. Panel C is a graded scaffold from Group C with central small pores of 350 to 500 μm and peripheral large pores of 600 to 800 μm. Panel D is a graded scaffold from Group D with central large pores of 600 to 800 μm and peripheral small pores of 350 to 500 μm.

Consistent with dissolution results obtained in vitro, FIG. 12 shows that one month after implantation in vivo, the regions of the scaffolds with smaller pores had also degraded more rapidly than had the regions with larger pores. The results demonstrate that architecture of the scaffolds can be used to guide spatial biodegradation in vivo and thus, among other things, control release of incorporated factors.

### Example 11

### Protein Loading of CaP Scaffolds

The effects of varying the loading method and of varying pore size of scaffolds on the elution profile of proteins was evaluated utilizing bovine serum albumin (BSA) and Bone Morphogenetic Protein-2 (BMP-2). The BSA was loaded onto the porous scaffolds in two ways, by a drop method and by an immersion method. In the dropping method, a BSA solution was pipetted directly into the porous scaffolds. In the immersion method, the porous scaffolds were immersed into a BSA protein solution having the same concentration as was used for the dropping method. The subsequent elution profile for the protein was then evaluated.

The drop method resulted in consistent BSA loading and elution profiles for porous scaffolds of all pore sizes. In contrast, the immersion method produced significant differences in loading and elution for porous scaffolds that was dependent on the pore size in the scaffold.

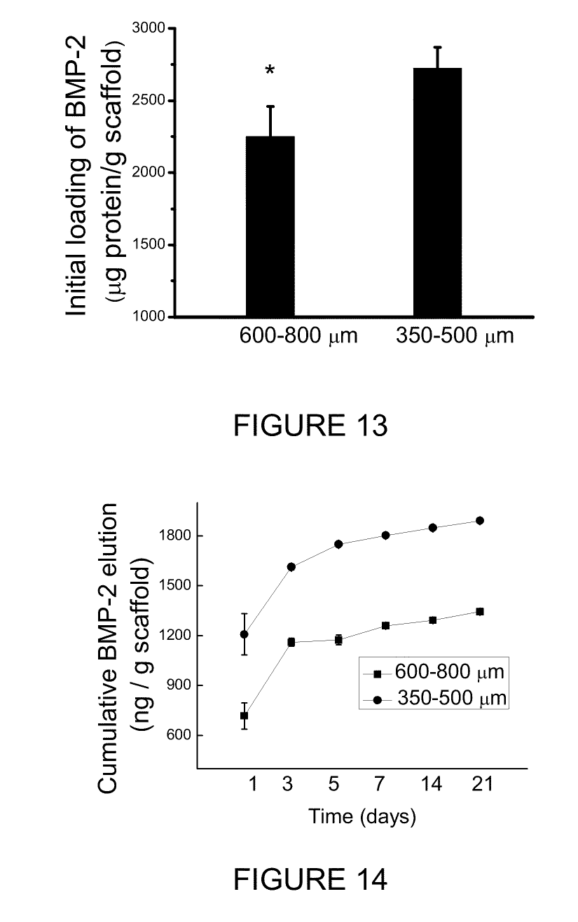

The immersion method was used to load BMP-2 onto the porous scaffolds. FIG. 13 shows that the immersion method resulted in a pore size dependent initial loading for BMP-2 that was similar to that for the loading of BSA. FIG. 14 shows that the elution profiles over a 21 day period can be regulated by varying scaffold pore size when using the immersion method of loading protein. Thus, temporally and spatially controlled release of bioactive agents such as growth factors and drugs by the disclosed ceramic scaffolds are feasible in some embodiments.

### Example 12

### CaP Scaffolds Loaded with BMP-2

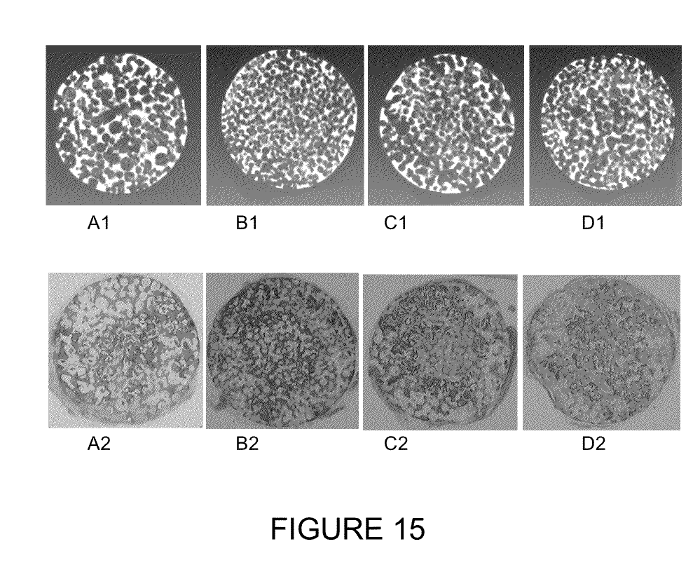

A study was performed to determine if varying the architecture of CaP scaffolds would have a temporal and/or spatial effect on BMP-2 induced osteogenesis. BMP-2 was loaded into the scaffolds by the immersion method as described above and the scaffolds were implanted subcutaneously into mice. One month after implantation, BMP-2 induced ectopic bone formation was evaluated by micro CT scan and histomorphometry. FIG. 15 shows the BMP-2 induced ectopic bone formation in the non-decalcified porous CaP scaffolds at one month after implantation. Micro CT images in panels A1, B1, C1, and D1 clearly demonstrate that the porous scaffolds are filled with substances. The histology pictures in panels A2, B2, C2, and D2 confirm that the substance filling the porous scaffolds is newly formed bone. When viewed at higher magnification (not shown), it was clear that the newly formed bone seamlessly contacts the scaffolds and fills the interconnective pores. Table 2 lists the histomorphometrical results of ectopic bone formation in porous β-TCP scaffolds at one month after implantation.

TABLE 2

| | Bone formation | Biodegradation |
|---|---|---|
| Scaffolds | rate (%) | rate (%) |
| Scaffolds with uniform 600-800 μm | 13.20 ± 3.88^{a} | 14.04 ± 2.48^{c} |
| pores (See FIG. 15A) | | |
| Scaffolds with uniform 350-500 μm | 8.62 ± 11.30 | 9.49 ± 4.22^{d,e} |
| pores (See FIG. 15B) | | |
| Graded scaffolds with central | 11.62 ± 4.55^{b} | 17.94 ± 2.52^{d} |
| 350-500 μm and peripheral | | |
| 600-800 μm pores (See FIG. 15C) | | |
| Graded scaffolds with central | 21.86 ± 3.21^{a,b} | 21.79 ± 2.65^{c,e} |
| 600-800 μm and peripheral | | |
| 350-500 μm pores (See FIG. 15D) | | |

Note:
1. Bone formation rate = new bone area/whole tissue area × 100;
2. Bone area is determined by quantitative histomorphometry;
3. Sample number = 3 samples per group;
4. a, b, c, d and e indicate significant differences (P < 0.05).

As shown in Table 2, graded scaffolds with central 600-800 μm pores and peripheral 350-500 μm pores exhibited significantly greater bone formation compared to uniform scaffolds with 600-800 lam pores (P=0.04089) and graded scaffolds with central 350-500 μm pores and peripheral 600-800 μm pores (P=0.03345). The uniform scaffolds with 350-500 μm pores did not exhibit significantly different bone formation as compared to uniform scaffolds with 600-800 μm pores (P=0.53853) and to graded scaffolds with central 350-500 μm pores and peripheral 600-800 μm pores (P=0.69125). These studies indicate that of the presently tested architectures, an optimum architecture for CaP scaffolds for induction of osteogenesis may be the graded scaffold with central large pores and peripheral small pores. Notably, in these studies the % new bone formation substantially offset % biodegradation rate of the implanted scaffold during the one-month period after implantation, and suggests that an implanted scaffold is maintained substantially intact at the implantation site long enough to allow bony tissue to grow in the scaffold.

### Example 13

### Scaffold-Aided Bone Healing

A representative porous CaP scaffold, with or without recombinant human BMP-2 (rhBMP-2), prepared as described above, was evaluated for the ability to enhance bone formation and healing using an accepted rabbit radius critical sized bone defect model. Porous CaP scaffolds loaded with BMP-2 were implanted into a 1.5 cm bone defect in the right radii of New Zealand rabbits, and porous CaP scaffolds without BMP were implanted into a similar defect in the left radii as a control.

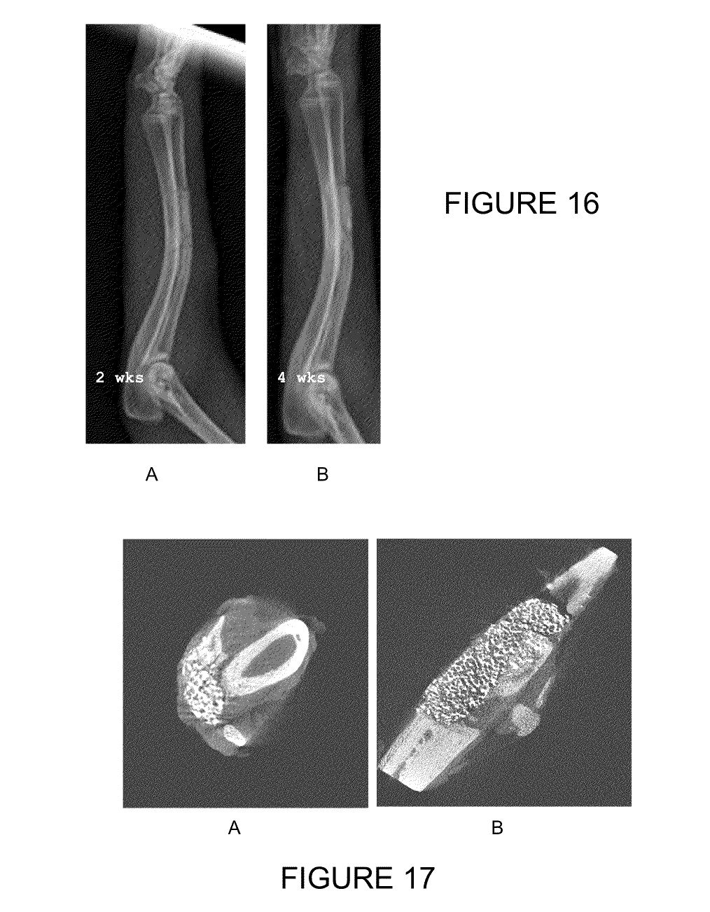

It was demonstrated that both the porous scaffolds in the presence and absence of BMP-2 aided bone healing as determined at one month after implantation. FIG. 16 shows the radiographic observation of scaffold-aided bone healing at 2 weeks (panel A) and one month (panel B) following implantation. As shown in FIG. 17, the micro CT images of scaffold-aided bone healing obtained one month after implantation, show new bone formation is visible among the pores of the scaffolds. Clearly, the implanted biodegradable scaffold is maintained substantially intact at the implantation site long enough to allow bony tissue to grow in the scaffold as the implanted ceramic article gradually biodegrades.

### Example 14

### Three-zone Graded Ceramic Scaffold Containing Fibers

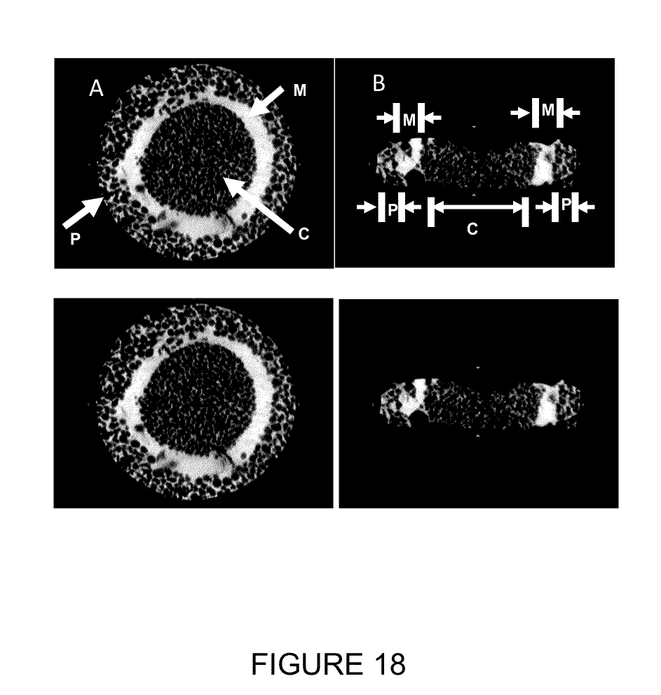

Referring now to FIG. 18, a three-zone graded CaP ceramic scaffold was constructed in a method similar to that described previously for the two-zone graded ceramic scaffolds except that it comprises three concentric zones. A central porous cylinder having a porosity of 80% with macropore diameters of 300-500 μm is identified in FIG. 18 as C. A middle cylinder (identified in FIG. 18 as M) that was denser with a porosity of 20% and a peripheral cylinder (identified in FIG. 18 as P) had a porosity of 80% porosity with macropores ranging from 600-800 μm in diameter. (A) is a coronal view of the 2D image. (B) is a sagital view of the 2D image. In addition, the biopolymer chitosan was incorporated to improve the torsion and bending strength of the composite scaffold. The chitosan biopolymer was infiltrated into the 3D porous ceramic network to form an integrated composite, using about 0.5 to about 1 wt % chitosan solution. Alternatively, the ceramic scaffold may be infiltrated with a PLLA solution.

A two-zone graded CaP ceramic scaffold was shown in preceding examples to have high compressive strength that is equivalent to that of long bones. However, a three-zone graded ceramic-polymer structure, with its more non-homogeneous nature, comprising a structure with a dense and stiff external layer, similar to that of compact bone, and increasing porosity toward the center, similar to what is seen in cancellous bone, provides a more natural bone-like structure. In addition, the presence of a central porous cylinder may be used to delivering growth factors and/or cells that may enhance osteointegration. Alternatively, a central porous channel may facilitate attachment of hardware during surgery, for example, when using screws, intramedullary nails and inserts as well as other devices. Likewise, the presence of a middle denser ceramic cylinder, in some embodiments, may provide high compression strength, comparable to human bone. Thus, a three-zone graded ceramic-polymer structure is expected to also have high bending and torsion mechanical strength that is equivalent to those of long bones. This method of making a ceramic-polymer article will provide further improved methods of generating materials for use in bone grafts for the repair of large load-bearing bone defects.

For various applications, the templates of different zones may be formed so as to have different porosities and pore sizes, and, in some cases, different slurries are cast into different zones. For example, in some cases a dense layer or pore-less layer or zone is desired. In another example, for preparing a three-zone article a template is prepared having centrally arranged beads and peripherally arranged beads, but the middle cylinder of the template is an empty space with no paraffin beads. After the slurry is cast into the negative template consisting of arranged beads and empty space, the seamlessly integrated porous/dense scaffold is treated by solvent extraction, as described above.

In an exemplary embodiment, a ceramic article prepared by solvent extraction as described above comprises an innermost zone having a porosity in the range of about 70% to about 100% and mean pore diameter in the range of about 1 μm to about 1 cm. The article also has an outermost zone with a porosity in the range of about 70% to about 90% and mean pore diameter in the range of about 1 μm to about 1 cm. Disposed between, and in contact with the innermost and outermost zones is a middle zone having a greater density than the other zones. For instance, a porosity of about 20%. In various applications, different sub-ranges within the above-stated pore size range are employed. For instance, mean pore diameters of about 1 μm-10 μm, 100 μm, 1 mm, 10 mm, 100 mm, 200 mm, 400 mm, 600 mm, 800 mm and 1 cm.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments of the invention have been shown and described, many variations and modifications thereof can be made by one skilled in the art without departing from the spirit and teachings of the invention. For example, although different exemplary embodiments may have been described as including one or more features providing one or more potential benefits, it is contemplated that the described features may be interchanged with one another or alternatively be combined with one another in the exemplary embodiments or in other alternative embodiments. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of making a ceramic article, comprising:
a) forming at least one ceramic composition into a defined shape comprising at least two zones with different porosity or pore size, wherein a second said zone surrounds a first said zone in at least two dimensions, each said ceramic composition containing a ceramic material and a liquid;
b) exposing the shaped ceramic composition(s) to a solvent in which the liquid is soluble, to extract the liquid from and thereby harden said shaped ceramic composition(s); and
c) solidifying the resulting hardened ceramic composition(s) to provide the ceramic article.

2. The method of claim 1, wherein, in a) said forming comprises casting at least one of said compositions onto a replica that is insoluble in the solvent.

3. The method of claim 2, wherein the replica is a negative replica corresponding to a said zone and comprising a sacrificial porogen comprising a multiplicity of discrete elements.

4. The method of claim 3, wherein in a), said forming comprises: causing said multiplicity of discrete elements to coalesce to a degree that corresponds to interconnectivity of pores of at least 70% in the ceramic article.

5. The method of claim 3, wherein in a), said forming comprises:
a₁) forming a first composition into a first zone; and
a₂) forming a second composition into a second zone that surrounds said first zone in at least two dimensions,
said first and second zones comprising respective first and second multiplicities of discrete elements wherein the elements of the first zone differ in size from the discrete elements of the second zone, to provide said ceramic article with graded porosity.

6. The method of claim 3, further comprising: after b) and before c),
b′) at least partially removing the sacrificial porogen from the hardened ceramic composition.

7. The method of claim 1, wherein, in b), said exposing comprises exposing the shaped ceramic composition to stepwise increases in solvent concentration.

8. The method of claim 1, wherein, in a), the liquid comprises water, and in b), the solvent comprises a liquid in which water is soluble.

9. The method of claim 1, wherein said ceramic material comprises calcium phosphate.

10. The method of claim 1, further comprising d) associating a polymer or a growth factor with the solidified ceramic article.

11. The method of claim 1, wherein in a), said forming comprises:
a₁) forming a first composition into a first defined zone;
a₂) forming a second composition into a second defined zone that surrounds said first defined zone in at least two dimensions;
a₃) forming a third composition into a third defined zone that surrounds said second defined zone in at least two dimensions.

12. A ceramic article comprising:
at least two zones comprising at least one ceramic material, wherein a second said zone surrounds a first said zone in at least two dimensions, at least two of said zones having different porosity or pore size, having solid struts between pores, and at least 70% pore interconnectivity.

13. The ceramic article of claim 12 having compressive strength equal to or exceeding that of cortical bone.

14. The ceramic article of claim 12, wherein said ceramic article is made by a process that comprises: a) forming at least two ceramic compositions into at least two defined zones wherein a second said zone surrounds a first said zone in at least two dimensions, each said ceramic composition containing a liquid; b) exposing the resulting at least two formed ceramic compositions stepwise to increasing concentrations of a solvent in which the liquid is soluble, to extract the liquid from and thereby harden said shaped ceramic compositions; and c) solidifying the resulting hardened ceramic compositions to provide the ceramic article having a defined shape.

15. The ceramic article of claim 12, wherein said at least two zones comprises a third said zone surrounding said second zone in at least two dimensions.

16. The ceramic article of claim 15, wherein said first zone comprises an innermost zone having a porosity in the range of about 70% to about 100% and mean pore diameter in the range of about 1 μm to about 1 cm;
said third zone comprises an outermost zone having a porosity in the range of about 70% to about 90% and mean pore diameter in the range of about 1 μm to about 1 cm; and
said second zone comprises a middle zone disposed between and in contact with said innermost and outermost zones and having a greater density than that of at least one of said innermost and outermost zones.

17. The ceramic article of claim 16, wherein
said innermost zone has a porosity in the range of about 70% to about 90% and a mean pore diameter in the range of about 300-500 μm,
said middle zone has a porosity of about 20%, and
said outermost zone has a porosity in the range of about 70% to about 90% and a mean pore diameter in the range of about 1 μm to about 2 cm.

18. The ceramic article of claim 12, wherein said ceramic article further comprises a polymer.

19. A ceramic article made by the method of claim 11.

20. A method of repairing a bone defect in an individual, comprising:
a) implanting into a defect of a bone within the individual the ceramic article of claim 12 configured as a biodegradable scaffold wherein said second zone has 70-90% porosity; and
b) allowing bony tissue to grow in the scaffold while said ceramic article gradually biodegrades.
